(19) United States
(12) Patent Application Publication
(10) Pub. No.: US 2020/0383679 A1
(43) Pub. Date: Dec. 10, 2020

(54) MULTIPLE ANCHOR DELIVERY SYSTEM AND METHOD

(71) Applicant: T.A.G. Medical Devices - Agriculture Cooperative Ltd., Kibbutz Gaaton (IL)

(72) Inventors: Aviram ALFIA, Karmiel (IL); Dima GUREVICH, Carmiel (IL); Hagay SITRY, Kibbutz Gesher HaZiv (IL); Roy ZILBERMAN, Qadarim (IL)

(73) Assignee: T.A.G. Medical Devices - Agriculture Cooperative Ltd., Kibbutz Gaaton (IL)

(21) Appl. No.: 16/767,137

(22) PCT Filed: Oct. 27, 2018
(86) PCT No.: PCT/IL2018/051298

§ 371 Date: May 27, 2020

Related U.S. Application Data
(60) Provisional application No. 62/591,132, filed on Nov. 27, 2017.

(51) Int. Cl. A61B 17/04

(52) U.S. Cl. CPC A61B 17/0401; A61B 2017/0464; A61B 2017/0409

(57) ABSTRACT

A multiple anchor delivery system, comprising: a sheath having a passageway extending therethrough; a pusher element having a distal end positioned within the passageway, the pusher element distal end sized and shaped to be displaced through the passageway; a first anchor disposed within the sheath, a portion of the first anchor distal to the pusher element; and a second anchor disposed within the passageway, proximal to the first anchor, the second anchor sized and shaped to be displaced along the passageway. The pusher element is sized and shaped to be displaced proximally to a position proximal to at least a distal portion of the second anchor. The pusher element is sized and shaped to be displaced distally by a second displacement length at least as long as the distance between the second anchor proximal end and a sheath distal end, the second displacement length sufficient to deploy the second anchor.

### RELATED APPLICATION/S

This application claims priority from U.S. Provisional Patent Application No. 62/591,132 to Alfia et al., filed on Nov. 27, 2017, the contents of which are hereby incorporated herein by reference in their entirety.

### FIELD AND BACKGROUND OF THE INVENTION

The present invention generally relates to soft tissue repair systems, for example, multiple anchor delivery systems.

It is known that various surgical procedures are performed in order to repair a deformed or torn soft tissue, such as meniscus, by means of suturing. There is a particular need for systems enabling delivery of several anchors during a single procedure.

U.S. Pat. Nos. 8,888,798 and 9,357,994, both to Smith & Nephew, Inc., each discloses a tissue repair device “wherein advance of the knob allows for engagement of the actuator with the first anchor and subsequent advancement of the first anchor.”

U.S. Pat. Nos. 9,498,203 and 9,549,725 to Smith & Nephew each disclose a tissue repair device including “a spring-loaded pusher” configured “for delivery of a flexible member to secure the tissue.”

U.S. Pat. No. 9,622,736 discloses a tissue repair device that “includes first and second tubular anchors having corresponding longitudinal passages. The tissue repair device includes corresponding first and second inserters. Each inserter has a shaft with a distal portion received in the longitudinal passage of the corresponding tubular anchor. A flexible strand couples the first and second anchors” (abstract).

Additional anchor repair devices are disclosed in U.S. Pat. Nos. 5,954,747; 6,306,156; 5,980,558; 5,993,459; 6,146,407; 6,595,911; 2003/0167072; US 2008/0167660; U.S. Pat. Nos. 9,249,266; 9,173,645; 5,236,445; 4,899,743; 4,946,468; 4,968,315; 5,002,550; 5,041,129; 5,123,914; 5,258,016; 5,372,604; 5,403,348; 5,417,712; 5,417,691; 5,626,614; 5,718,717; 5,954,747; 6,554,852; 6,511,498; 5,403,348; 7,857,830; 7,905,903; 7,601,165; 8,128,658; 7,959,650; 8,771,314; 8,298,262; 8,221,454; US 2014/0039552; U.S. Pat. Nos. 8,652,172; 8,828,053; 9,463,011; and 9,622,738.

### SUMMARY OF THE INVENTION

The present invention seeks to provide an improved multiple anchor delivery system.

There is thus provided in accordance with some embodiments of the present invention a multiple anchor delivery system, including a driving unit and a hollow needle extending distally therefrom; a pusher element operatively coupled with the driving unit and extending distally therefrom within the hollow needle; a first solid anchor disposed within the hollow needle, distally with respect to the pusher element; and a second tubular anchor being threaded onto the pusher element.

Preferably, at least one of the first and second anchors is bio-absorbable. Further preferably, the driving unit includes a linearly displaceable trigger button. Still further preferably, the trigger button is configured for interaction with a pinion. The pinion is configured for interaction with a rack, which linearly displaces the pusher element within the hollow needle. Preferably, the pusher element includes a releasing feature for releasing the second anchor during retraction of the pusher element. Optionally, instead of a linearly displaceable trigger button, the driving unit may be actuated by a rotatable roller configured for interaction with a rack.

In accordance with some embodiments of the present invention, a method of delivering multiple anchors into a tissue of a patient, including delivering a hollow needle into a tissue; displacing a trigger button in a first direction to deploy a first anchor into the tissue, displacing the trigger button in a second direction, which is opposite to the first direction, to load the second anchor; and displacing the trigger button in the first direction to deploy the second anchor.

Preferably, the trigger button is displaced proximally to deploy at least one of the first and the second anchor. Further preferably, the trigger button is displaced distally to load the second anchor. Still further preferably, a tactile indication for deployment of the anchor or for loading of the anchor is provided to a user during displacement of the trigger button.

According to an aspect of some embodiments of the present invention there is provided a multiple anchor delivery system, comprising: a sheath having a proximal end and a distal end and having a passageway extending therethrough; a pusher element having at least a distal end positioned within the passageway, the pusher element distal end sized and shaped to be displaced through the passageway; a first anchor disposed within the sheath, at least a portion of the first anchor positioned distally with respect to the pusher element; and a second anchor disposed within the sheath, proximally with respect to the first anchor, the second anchor disposed within the passageway and, the second anchor sized and shaped to be displaced along the passageway; the pusher element sized and shaped to be displaced in a proximal direction to a position proximal to at least a distal portion of the second anchor; and the pusher element sized and shaped to be displaced in the distal direction, the displacement of the pusher element having a second displacement length at least as long as the distance between the proximal end of the second anchor and the sheath distal end, the second displacement length sufficient to deploy the second anchor.

According to some embodiments of the invention, the second anchor overlaps axially with the pusher element.

According to some embodiments of the invention, the second anchor is tubular.

According to some embodiments of the invention, the pusher element sized and shaped to be displaced in a distal direction, into contact with the first anchor, the displacement having a first displacement length sufficient to deploy the first anchor.

According to some embodiments of the invention, the system further includes a barrier that narrows the passageway, the barrier obstructing a proximal displacement of the second anchor.

According to some embodiments of the invention, the second anchor is mounted on the pusher element.

According to some embodiments of the invention, the pusher element distal end includes a widened tip.

According to some embodiments of the invention, the pusher element extends through an interior of the second anchor.

According to some embodiments of the invention, the second anchor is compressible in a lateral direction.

According to some embodiments of the invention, the second anchor is compressible in a lateral direction, the pusher element displaceable in a proximal direction to a position proximal to the second anchor.

According to some embodiments of the invention, the first anchor includes a blind bore at its proximal end, the pusher element distal end sized and shaped to fit into the blind bore.

According to some embodiments of the invention, the pusher element distal end includes a retaining portion for temporarily retaining the second anchor thereon.

According to some embodiments of the invention, the retaining portion is a recess for retaining a second anchor.

According to some embodiments of the invention, the pusher element is provided with a compressible distal portion.

According to some embodiments of the invention, the first anchor is tubular.

According to some embodiments of the invention, at least one of the first and second anchor is provided with a channel along at least a portion of its length, the channel having a passageway sized for passage of a suture portion therethrough.

According to some embodiments of the invention, the sheath includes a hollow needle.

According to some embodiments of the invention, the sheath has a lateral opening at the distal end thereof, the opening large enough to for at least two suture elements to pass therethrough.

According to some embodiments of the invention, the sheath has a lateral opening at the distal end thereof, the second anchor positioned proximal to the lateral opening prior to deployment of the second anchor.

According to some embodiments of the invention, the sheath has a sharpened tip at its distal end.

According to some embodiments of the invention, the sheath distal end is curved.

According to some embodiments of the invention, the system further includes an actuator mechanically coupled to the pusher element, movement of the actuator affecting linear displacement of the pusher element along the passageway.

According to some embodiments of the invention, the system further includes a driving unit for displacing the pusher element through the passageway, wherein the actuator is mechanically coupled with the driving unit.

According to some embodiments of the invention, displacement of the actuator in a first direction affects via the driving unit the displacement of the pusher element in a second direction, the second direction opposite to the first direction.

According to some embodiments of the invention, the first direction is one of a proximal direction and a distal direction.

According to some embodiments of the invention, the system further includes a housing for housing the pusher element and at least a proximal portion of the sheath, the housing having a semicircular portion at a distal end thereof and a suture holder having a semicircular arm at a proximal end thereof, the arm and the housing portion together forming a passageway sized for passage therethrough of a suture.

According to some embodiments of the invention, the system further comprises a locking element for preventing the proximal displacement of the pusher element.

According to some embodiments of the invention, the system further comprises: a driving unit for displacing the pusher element through the passageway; wherein the locking element obstructs a path of movement of the driving unit.

According to some embodiments of the invention, the first and second anchors are entirely housed within the sheath prior to their deployment.

According to an aspect of some embodiments of the present invention there is provided a multiple anchor delivery system, comprising: a sheath having a proximal end and a distal end and having a passageway extending therethrough; first and second pusher elements disposed inside the sheath, each pusher element having at least a distal end positioned within the passageway, the distal end of each the pusher element sized and shaped to be displaced through the passageway; a first anchor disposed within the sheath, at least a portion of the first anchor positioned distally with respect to the first pusher element; a second anchor disposed within the sheath, proximally with respect to the first anchor; the first pusher element displaceable in a distal direction, into contact with the first anchor, the displacement having a displacement length sufficient to deploy the first anchor; the second pusher element displaceable in a distal direction, into contact with the second anchor, the displacement having a displacement length sufficient to deploy the second anchor.

According to an aspect of some embodiments of the present invention there is provided a multiple anchor delivery system, comprising: a sheath having a proximal end and a distal end and having a passageway extending therethrough; a pusher element having at least a distal end positioned within the passageway, the pusher element distal end sized and shaped to be displaced through the passageway; a first anchor disposed within the sheath, at least a portion of the first solid anchor positioned distally with respect to the pusher element; and a second anchor disposed within the sheath, proximally with respect to the first anchor, the second anchor mounted on the pusher element.

According to some embodiments of the invention, the second anchor is tubular.

According to some embodiments of the invention, the pusher element distal end includes a retaining portion for temporarily retaining the second anchor thereon.

According to some embodiments of the invention, the pusher element extends through an interior of the second tubular anchor.

According to an aspect of some embodiments of the present invention there is provided a method of delivering multiple anchors into a tissue of a patient, comprising: delivering a sheath through a tissue such that a sheath distal end penetrates the tissue; displacing an actuator in a proximal direction to deploy a first anchor out of the sheath and through the tissue; displacing the actuator in a distal direction to position a proximal end of a pusher element at least partly proximally to the second anchor; and displacing the actuator in the proximal direction to engage the second anchor and to deploy the second anchor out of the sheath.

According to some embodiments of the invention, wherein the first anchor is provided with a suture portion, the suture portion deployed out of the sheath and through the tissue with deployment of the first anchor.

According to an aspect of some embodiments of the present invention there is provided a method of operation of a device for deployment of multiple anchors, comprising: first displacement of a pusher element through a sheath in a distal direction; engagement of the pusher element with a first anchor located in the sheath, the first displacement having a first displacement length sufficient to deploy the first anchor out of the sheath; displacement of the pusher element through the sheath in a proximal direction, to a position proximal to a second anchor; second displacement of the pusher element through the sheath in the distal direction; and engagement of the pusher element with the second anchor during the second displacement, the second displacement having a second displacement length sufficient to deploy the second anchor out of the sheath.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a simplified sectional view illustration of an exemplary multiple anchor delivery system and an enlargement view of a distal end thereof, shown in an initial operative orientation, in accordance with some embodiments of the invention;

FIG. 2 is a simplified sectional view illustration of the multiple anchor delivery system and an enlargement view of a distal end thereof, shown in a first anchor deployment operative orientation, in accordance with some embodiments of the invention;

FIG. 3 is a simplified sectional view illustration of the multiple anchor delivery system and an enlargement view of a distal end thereof, shown in a retraction operative orientation, in accordance with some embodiments of the invention;

FIG. 4 is a simplified sectional view illustration of the multiple anchor delivery system and an enlargement view of a distal end thereof, shown in a second anchor deployment operative orientation, in accordance with some embodiments of the invention;

FIG. 5 is a perspective view of an exemplary multiple anchor delivery system, in accordance with some embodiments of the invention;

FIG. 6 is an exploded view of the device shown in FIG. 5, in accordance with some embodiments of the invention;

FIGS. 7A-E are respective left side view, perspective view from the left side, right side view, bottom view, and end view of a right half of an exemplary housing of the device shown in FIG. 6, in accordance with some embodiments of the invention;

FIGS. 8A-E are respective perspective, top, side, bottom, and front views of a exemplary rack, in accordance with some embodiments of the invention;

FIG. 9A is a perspective view of an exemplary roller, in accordance with some embodiments of the invention;

FIGS. 9B-C are side views of exemplary rollers in accordance with alternative embodiments of the invention;

FIGS. 10A-D are respective perspective, top, side, and end view of an exemplary bobbin, in accordance with some embodiments of the invention;

FIGS. 11A-D are respective perspective, front, side, and top views of an exemplary locking element, in accordance with some embodiments of the invention;

FIGS. 12A-E are respective perspective, side, top, end, and cross-sectional views of an exemplary needle length delimiter, in accordance with some embodiments of the invention;

FIG. 13A is a perspective view of an exemplary sheath, with FIG. 13D showing an enlargement of a distal end thereof, in accordance with some embodiments of the invention;

FIGS. 13B-C are respective cross-sectional and top views of the sheath shown in FIG. 13A, in accordance with some embodiments of the invention;

FIG. 14A is a side view of an exemplary pusher element, with FIG. 14B showing an enlargement of a distal portion thereof, in accordance with some embodiments of the invention;

FIGS. 15A-C are respective perspective, side, and top views of an exemplary suture holder in accordance with some embodiments of the invention;

FIGS. 15D-E are respective side and top views of an exemplary cannula in accordance with some embodiments of the invention;

FIGS. 16A-C are respective perspective, cross-sectional, and top views of exemplary first and second anchors with suture arranged therethrough, in accordance with some embodiments of the invention;

FIGS. 17A-D are a side-sectional view of an exemplary system according to some embodiments of the invention with enlargements of portions thereof, an enlargement of a distal portion of the system, a top view of the system, and a side view of the system, respectively, prior to use, in accordance with some embodiments of the invention;

FIGS. 18A-D are a side-sectional view of the system shown in in FIG. 17A, with an enlargement of a distal portion of the system shown in FIG. 18B, a top view of the system, and a side view of the system, respectively, after insertion of a distal end thereof through a tissue, in accordance with some embodiments of the invention;

FIGS. 19A-D are a side-sectional view of the system shown in FIG. 18A with an enlargement of a distal portion of the system shown in FIG. 19B, a top view of the system, and a side view of the system, respectively, after deployment of a first anchor through the tissue, in accordance with some embodiments of the invention;

FIGS. 19E-H are a side-sectional view of the system shown in FIG. 19A with an enlargement of a distal portion of the system shown in FIG. 19F, a top view of the system, and a side view of the system, respectively, after retraction of the pusher element, in accordance with some embodiments of the invention;

FIGS. 20A-D are a side-sectional view of the system shown in FIG. 19E with an enlargement of a distal portion of the system, a top view of the system, and a side view of the system, respectively, after withdrawal of the device from the tissue, in accordance with some embodiments of the invention;

FIGS. 20E-G are a side-sectional view of the system shown in FIG. 19E, an enlargement of a distal portion of the system shown in FIG. 19F, and a top view of the system, in accordance with some embodiments of the invention;

FIGS. 21A-D are a side-sectional view of the system shown in FIG. 20A with an enlargement of a distal portion of the system, a top view of the system, and a side view of the system, respectively, after deployment of a second anchor through the tissue, in accordance with some embodiments of the invention;

FIGS. 22A-C are a side-sectional view of the system shown in FIG. 21A with an enlargement of a distal portion of the system, and a top view of the system, respectively, after withdrawal of the device distal end from the tissue, in accordance with some embodiments of the invention;

FIGS. 23A-B are respective front and side views of exemplary anchors after attachment to a tissue, before tightening of the suture material, in accordance with some embodiments of the invention;

FIGS. 23C-D are respective side and front views of the anchors shown in FIG. 23A, after tightening of the small suture loop, in accordance with some embodiments of the invention;

FIGS. 23E-G are respective side, back, and perspective views of the anchors shown in FIG. 23D after tightening of the large suture loop, in accordance with some embodiments of the invention;

FIG. 24 is a schematic illustration of an exemplary method of delivering multiple anchors into a tissue of a patient, in accordance with some embodiments of the invention; and

FIG. 25 is a schematic illustration of an exemplary method of operation of a device for deployment of multiple anchors, in accordance with some embodiments of the invention.

### DESCRIPTION OF SPECIFIC EMBODIMENTS OF THE INVENTION

The present invention, in some embodiments thereof, relates to a soft tissue repair system, and, more particularly, but not exclusively, to a multiple anchor delivery system.

A multiple anchor delivery system and method is disclosed herein, which is particularly useful for repairing soft tissue, such as meniscus for example, but not limited to this particular surgical procedure. An aspect of some embodiments of the invention relates to deploying multiple anchors using a sheath. In some embodiments of the invention, two anchors are deployed from the sheath, sequentially, by a single pusher element, with one anchor being in contact with the pusher element and a second anchor being loaded into the sheath and located to a side of the pusher element. Optionally, the second anchor is mounted on and/or around the pusher element.

In some embodiments of the invention, retraction of the pusher element after deployment of the first anchor repositions the second anchor relative to the pusher element so that subsequent distal advancement of the pusher element will deploy the second anchor from the sheath.

In some embodiments of the invention, when the pusher element is retracted in a proximal direction, the second anchor is pulled back with the pusher element until the second anchor encounters an interfering geometry and is prevented from further retraction. In this way the pusher element is moved distally relative to the second anchor.

Exemplary embodiments of the invention comprise a system and method for deploying multiple anchors through a tissue of a patient. The system and method may be utilized, for example, in the repair of torn meniscus tissue, by deploying first and second anchors through the torn tissue portions and optionally tightening the anchors against the tissue portions, thereby potentially holding the separated tissue portions together so that they may mend together. According to some embodiments, the system includes a device including a sheath having a passageway extending therethrough. A pusher element is positioned in the passageway and displaceable relative to the sheath, in a distal direction and a proximal direction.

An aspect of some embodiments of the invention relates to deploying first and second implants in the form of anchors from a sheath. The first and second anchors are sized and shaped to be positioned entirely within the passageway of a sheath, the first anchor positioned distally relative to the pusher element and the second anchor positioned proximally relative to the first anchor. Optionally, the first and second anchors are housed entirely within the sheath prior to deployment of the anchors.

A length of suture material may extend through the first and second anchors, forming a small loop and a large loop extending between the anchors. The small loop may extend from approximately the distal end of the first anchor to approximately the proximal end of the second anchor, and the free end may extend from approximately the proximal end of the first anchor, proximally through the device.

An aspect of some embodiments of the invention relates to deploying multiple anchors utilizing a sheath provided with a lateral opening such as, for example, a slot, at its distal end portion, the slot being wide enough for the small and large suture loops to pass therethrough. Optionally, the second anchor is positioned proximal to the lateral opening prior to deployment of the second anchor.

The device may include a tubular suture holder for retaining the suture material within a limited distance from the needle, thereby potentially preventing tangling of the suture material when the anchors are moved through the passageway.

According to some embodiments of the invention, the pusher element may be displaceable by linear displacement of a driving mechanism. The driving mechanism may be displaced by linear displacement of an actuator in the form of a trigger button. Alternatively, in some embodiments, the driving mechanism may be displaceable by rotation of a roller.

The actuator is movable in a distal direction and a proximal direction, the actuator coupled to the driving mechanism which is coupled to the pusher element. The coupling is arranged so that movement of the actuator in a distal direction results in displacement of the driving mechanism in a proximal direction, and that movement of the actuator in a proximal direction results in displacement of the driving mechanism in a distal direction.

The sheath may be inserted through a tissue such as, for example, a torn meniscus. The sheath should be inserted through the tissue such that the sheath distal end exits the tissue before deployment of the anchors is performed.

Once in position through the tissue, the device may be actuated by moving the actuator in a proximal direction, the pusher element is displaceable in a distal direction through the sheath so that it contacts the first anchor, the amount of displacement of the pusher element sufficient to deploy the first anchor from the sheath.

A barrier is positioned within the passageway at a location proximal to the second anchor, the barrier optionally in the form of a narrowing of the passageway so that it interferes with proximal displacement of the second anchor. The position of the second anchor within the sheath may or may not be affected by distal or proximal movement of the pusher, depending on the location of the second anchor relative to the barrier and the direction of movement of the pusher element.

After the first anchor has been deployed, the pusher element is displaceable through the passageway in a proximal direction to a position proximal to at least a portion of the second anchor. As the pusher element moves proximally, the second anchor may be prevented from also moving proximally once it contacts the barrier in the passageway.

The needle may be removed from the tissue and reinserted through the tissue at another location where it is desired to deploy the second anchor. When the device is actuated by again moving the actuator in a proximal direction, the pusher element is then displaceable distally through the sheath so that is contacts the second anchor, the amount of displacement of the pusher element sufficient to deploy the second anchor from the sheath.

After the second anchor has been deployed, the suture loops may be tightened, to tighten the anchors against the tissue. The anchors in position may hold the torn tissue portions together so that they may heal.

An aspect of some embodiments of the invention relates to deploying first and second anchors from a sheath, where the first anchor may be solid and the second anchor may be hollow. A pusher element may be displaced proximally and distally within the sheath, the pusher element being insertable through the second anchor. After deployment of the first anchor, the proximal displacement of the pusher element is effective to move the pusher element to a position proximal to the second anchor, wherein the second anchor is effectively loaded and ready for deployment. Upon subsequent distal displacement of the pusher element, the pusher element distal end contacts a proximal portion of the second anchor and deploys the second anchor out of the sheath. Optionally, the pusher element has a distal end wider than the second anchor proximal end, the pusher element distal end optionally being compressed when the when inserted through the second anchor. Optionally, the second anchor has a compressible portion which is decompressed upon retraction of the pusher element to a location proximal to the second anchor.

An aspect of some embodiments of the invention relates to deploying first and second anchors from a sheath, where the second anchor may be solid, and is optionally compressible. Optionally, the second anchor is laterally compressible. The second anchor may be compressed by the pusher element such that it is positioned alongside the pusher element. Upon retraction of the pusher element, the pusher element may slide past the second anchor, to position the second anchor distally relative to the pusher element. The second anchor may then be deployed by distal displacement of the pusher element. Optionally, the second anchor is compressible and, when decompressed, has an external diameter equal to or greater than the inner diameter of the sheath. In some embodiments, at least one of the first and second anchors may be hollow at least along a portion of its length, the anchor radially compressed by the sheath until deployed out of the sheath by the pusher element.

An aspect of some embodiments of the invention relates to a system and method for deploying first and second anchors, where the pusher element includes a recess for retaining the second anchor. Optionally, the recess may be a notch sized and shaped to temporarily contain the second anchor. Optionally, the second anchor may be positioned within the recess such that the second anchor is at least mostly surrounded by the pusher element. Optionally, the second anchor may be positioned within the recess such that the second anchor is surrounded on at least three sides by the pusher element. After deployment of the first anchor, the pusher element is retracted, which is effective to position the second anchor distally relative to the pusher element. The second anchor may then be deployed by distal displacement of the pusher element.

An aspect of some embodiments of the invention relates to deploying multiple anchors, the first and second anchors deployed by respective pusher elements, the pusher elements optionally working in parallel within a single sheath.

In accordance with some embodiments, the system may be provided with an actuator which is displaceable proximally to deploy the first anchor. Optionally, the actuator may be a trigger button which may be linearly displaceable. Alternatively, the actuator may be a roller which may be rotatable. In embodiments there is the potential advantage that a finger of a user moves in a first direction and the anchor is deployed in the second direction opposite to the first direction. For example, a user may actuate a device by a movement of a trigger button in a proximal direction, which results in deployment of an anchor in a distal direction out of the sheath.

In embodiments, the system may be provided with a locking mechanism which prevents inadvertent or premature deployment of the anchors.

An aspect of some embodiments of the invention relates to deploying multiple anchors using a sheath including an opening such as, for example, a slot, at a distal portion thereof. Suture portions provided on or through the anchors may be allowed to extend out of the opening, thereby potentially preventing tangling of the suture portions. Optionally, at least a portion of the second anchor is disposed proximal to the opening. Optionally, the entire length of the second anchor is disposed proximal to the opening.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Potential Advantages

A system and/or method in accordance with some embodiments of the present invention can provide one or more of a number of potential advantages not realized by the prior art. For example, the provision of a trigger button/roller, for example, as discussed herein may provide easy, one-handed actuation of the system by simple mechanical components. In addition, the amount of force required to be exerted on the trigger button/roller is substantially the same as the amount of force with which each anchor is deployed. This can allow a user to have control over the deployment of the anchors which is not possible in some prior art devices such as, for example devices wherein deployment of the anchors is by a spring-loaded mechanism.

Additionally, the provision of a system wherein finger movement of the user in a first direction results in displacement of the pusher element in a second direction, together with the provision of a safety latch/lock for example, as described herein, may prevent premature/inadvertent deployment of anchor(s).

Further, the ergonomic shape of the handle can allow the needle to be more easily maneuverable through the tissue, which may result in more accurate positioning of the anchors after deployment. Also, the ergonomic shape and operation of the handle and actuating mechanism, in the form of a trigger button or roller, together with engageable pusher driving unit results in less pressure required to deploy the anchors as compared with prior art devices.

Yet further, the provision of a slot in the needle through which the loops extend may allow for easier deployment of the anchors, as the loops/sutures may not interfere with movement of the anchors through the passageway, and the provision of a slot through which the loops extend may prevent tangling of suture material as the anchors are deployed from the needle.

Yet further, as in any device to be inserted into the human body, there is a limited amount of space within which to work. Having a pusher element inserted through the second anchor may save space within the device.

Yet further, provision of a bobbin on which suture material is wound may prevent tangling of the suture material within the housing.

These and other potential advantages may be apparent to persons skilled in the art.

These and other aspects of some embodiments of the invention are described herein, with reference to the accompanying drawings.

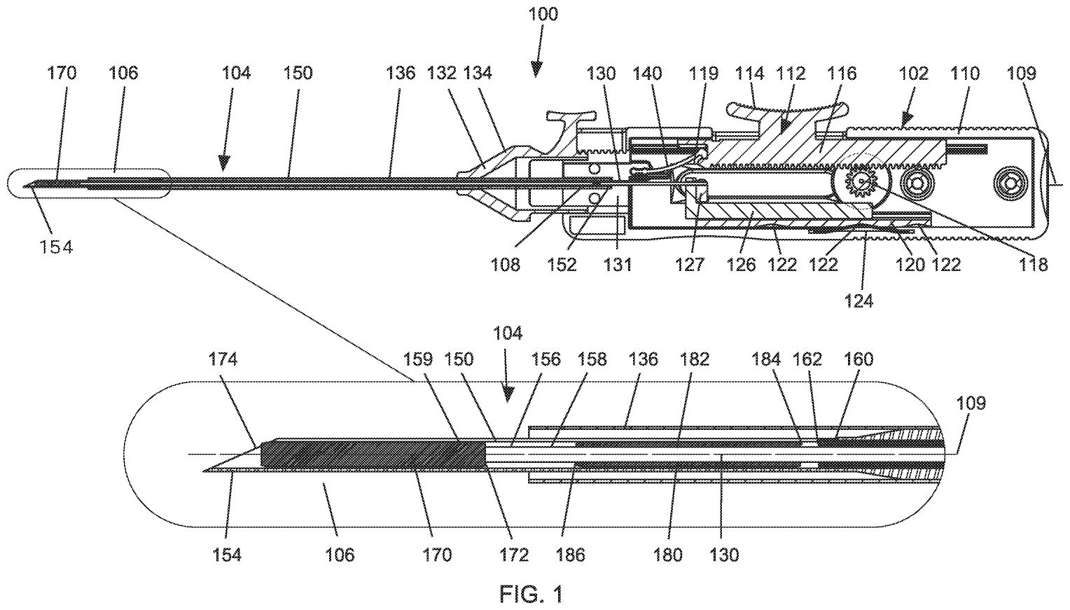

Reference is now made to FIG. 1, which is a simplified sectional view illustration of the multiple anchor delivery system and an enlargement view of a distal end thereof, shown in an initial operative orientation.

A system for delivery of multiple anchors in accordance with some embodiment is shown in FIGS. 1-4. The system shown includes a sheath having a passageway therethrough, a pusher element displaceable within the passageway, and first and second anchors displaceable by the pusher element along the passage and deployable from the passageway.

It is seen in FIG. 1 that a multiple anchor delivery system in accordance with some embodiments optionally includes a handle mechanism 102 and a delivery assembly which in the shown embodiment is a needle assembly 104, which is connected to the handle mechanism 102. The needle assembly 104 has a distal end 106 and a proximal end 108, which is connected to the handle mechanism 102. The handle mechanism 102 and the needle assembly 104 are optionally arranged along a mutual longitudinal axis 109.

The handle mechanism 102 has a housing 110, which is optionally made from plastic by injection molding. The housing 110 defines a handle which may be grippable by a user and is configured to contain the mechanism responsible for delivery of anchors through the needle assembly 104.

It is particularly seen in FIG. 1 that, according to the embodiment shown, a trigger button 112 resides partially within the housing 110, whereas the trigger button 112 has a gripping portion 114 for use by the finger of the user and an elongated toothed portion 116 for interaction with a pinion 118. Optionally, a distally extending projection 119 defining a shoulder may be formed at the distal end of the toothed portion 116. Alternatively, in some embodiments, a proximally extending projection defining a shoulder may be formed at the distal end of the toothed portion 116.

The pinion 118 also interacts with a rack 120. The rack 120 optionally has a first wall, which optionally includes a plurality of indents 122, for interaction with an optional leaf spring 124. The rack 120 further has a second wall, which is configured for slidable mounting of a support element 126 thereon. The rack 120 optionally has a projection 127 that is configured for fixed connection to a pusher element 130, which is configured to displace the anchors throughout the needle assembly 104. It is appreciated that the pusher element 130 is optionally solid and optionally made of stainless steel. Optionally, the pusher element 130 is not solid and may be optionally made of other material(s).

It is seen that the rack 120 is optionally disposed in parallel and may be spaced from elongated toothed portion 116 of trigger button 112.

It is seen in FIG. 1 that the support element 126 is optionally slidably coupled with the pusher element 130.

A support hub 131 is optionally formed within housing 110 of handle mechanism 102.

A needle depth limiter 132 is optionally connected to the support hub 131. The needle depth limiter 132 optionally includes a hub portion 134, which is rigidly coupled to the housing 110 and an elongated hollow cannula 136, extending along longitudinal axis 109.

It is further seen in FIG. 1 that a optionally flexible safety latch 140 is coupled to the support hub 131 and extends proximally thereof. It is seen that in this initial operative orientation the safety latch 140 is optionally supported against the shoulder formed by projection 119.

The needle assembly 104 includes a hollow needle 150, having a proximal end 152 and a distal sharpened end 154. The hollow needle 150 also defines an inner surface 156 and an interior volume 158. It is appreciated that the hollow needle 150 is optionally made of stainless steel.

It is seen particularly in the enlargement view of FIG. 1 that the pusher 130 is positioned within the interior volume 158 of needle 150 and extends distally from the proximal end 152 towards the distal end 154 of needle 150. The pusher 130 optionally defines a distally facing end surface 159.

An anchor stopper 160 is optionally located within the interior volume 158 of needle 150, at the proximal portion of the needle 150 and optionally defines a distally facing surface 162.

It is a particular feature of some embodiments of the present invention that a first anchor 170 is optionally solid and is disposed within the interior volume 158 of needle 150 distally to the pusher 130. The first anchor 170 has a proximal end 172 and a distal end 174, and it is seen that proximal end 172 may abut the distally facing end surface 159 of the pusher 130 in this initial operative orientation. It is noted that the first anchor is optionally soft, made of Polyethylene or Polypropylene. It is appreciated that alternatively, the anchor can be rigid. It is additionally noted that the first anchor 170 may be bio-absorbable.

It is a further particular feature of some embodiments of the present invention that a second anchor 180 is disposed proximally with respect to the first anchor 170 and the second anchor is optionally tubular, defining an inner surface 182. It is noted that the second anchor is optionally soft, made of Polyethylene or Polypropylene. It is appreciated that alternatively, the anchor can be rigid. It is additionally noted that the second anchor 180 may be bio-absorbable.

The second anchor 180 is threaded over the pusher 130, so that the inner surface 182 of the second anchor engages the outer surface of the pusher 130. The second anchor 180 has a proximal end 184 and a distal end 186. The proximal end 184 of the second anchor 180 is optionally disposed in the vicinity or abuts the distally facing surface 162 of the anchor stopper 160. The distal end 186 of the second anchor 180 is disposed distally to the proximal end 172 of the first anchor 170. Alternatively, in some embodiments, the distal end 186 of the second anchor 180 is disposed proximally to the proximal end 172 of the first anchor 170, for example, as shown in FIG. 1.

It is seen that the distal end of elongated hollow cannula 136 of the needle depth limiter 132 is disposed proximally of the distal sharpened end 154 of the hollow needle 150. Since the outer diameter of the cannula 136 is substantially larger than the outer diameter of the hollow needle 150, the penetration depth of the needle 150 into the tissue of the patient is limited to the extent to which the needle projects distally with respect to the distal end of cannula 136.

It is seen in FIG. 1 that in this initial operative orientation, trigger button 112 is disposed in an intermediate position. In this position, the rack 120 is also disposed in an intermediate position, in which the leaf spring 124 is seated within one of the indents 122 formed in rack 120.

It is a particular feature of some embodiments of the present invention that actuation of the trigger button 112 optionally affects displacement of the pusher 130 in the following manner: The user places his finger on gripping portion 114 and displaces the trigger button 112 proximally, thereby elongated toothed portion 116 of the trigger button 112 activates pinion 118, which in turn displaces the rack 120 distally due to the interaction between the rack 120 and the pinion 118. Since the pusher 130 is connected to rack 120, the pusher 130 is thereby also displaced distally.

During displacement of the rack 120, the leaf spring 124 is being positioned within a subsequent one of the indents 122, thereby potentially providing tactile indication to the user, indicating that the trigger button 112 is positioned in a different one of its operative orientations.

It is a further particular feature of some embodiments of the present invention that in this initial operative orientation the safety latch 140 is supported against the shoulder formed by projection 119 of the trigger button 112, which prevents distal displacement of the trigger button 112. This is a safety feature intended to prevent inadvertent deployment of anchor from the system 100. In this initial operative orientation, the trigger button 112 can only be displaced in a proximal direction, which is not intuitive for the user.

It is seen in FIG. 1 that in this initial operative orientation, the first anchor 170 is optionally disposed at the vicinity of the distal sharpened end 154 of the hollow needle 150, whereas the proximal end 172 of the first anchor 170 optionally abuts the distally facing end surface 159 of the pusher 130. The second anchor 180 is proximally spaced apart from the first anchor 170 in this initial operative orientation and is threaded over the outer surface of the pusher 130. The proximal end 184 of the second anchor 180 is optionally slightly distally spaced apart from distally facing surface 162 of the anchor stopper 160.

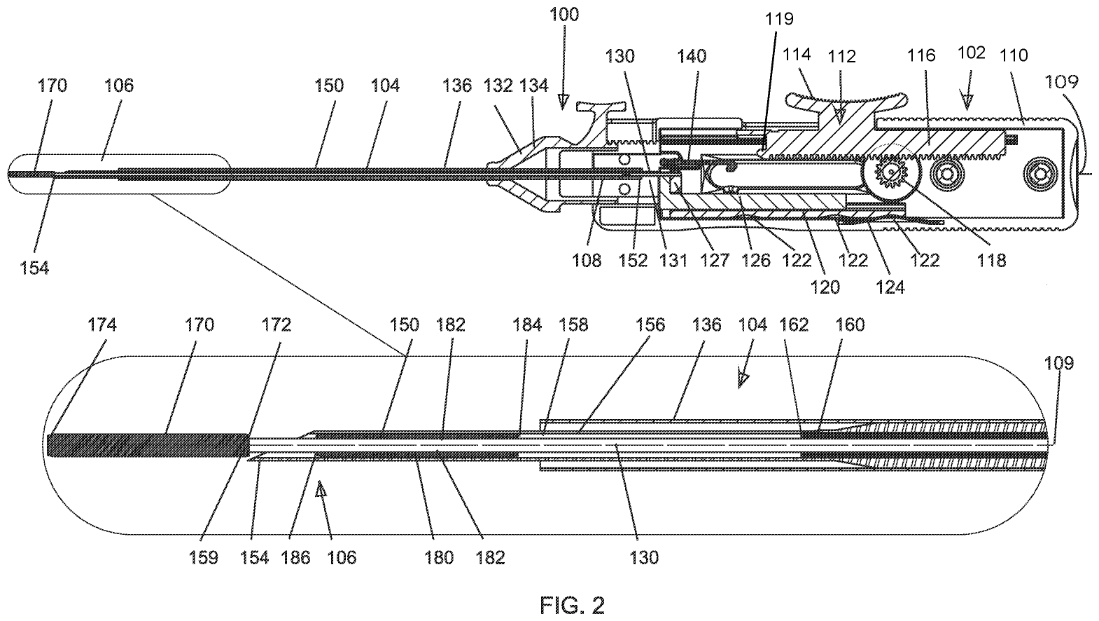

Reference is now made to FIG. 2, which is a simplified sectional view illustration of the multiple anchor delivery system 100 and an enlargement view of a distal end thereof, shown in a first anchor deployment operative orientation.

It is seen in FIG. 2 that in this first anchor deployment operative orientation, trigger button 112 is disposed in a proximal position. In this position, the rack 120 is disposed in a distal position, in which the leaf spring 124 is seated within another one of the indents 122 formed in rack 120.

The user displaced the trigger button 112 proximally, thereby elongated toothed portion 116 of the trigger button 112 activated pinion 118, which in turn displaced the rack 120 distally due to the interaction between the rack 120 and the pinion 118. Since the pusher 130 is connected to rack 120, the pusher 130 was thereby also displaced distally.

During displacement of the rack 120, the leaf spring 124 is being positioned within a subsequent one of the indents 122, thereby potentially providing tactile indication to the user, indicating that the trigger button 112 is now positioned in the proximal position.

It is a further particular feature of some embodiments of the present invention that in this first anchor deployment operative orientation, the safety latch 140 disengaged from the shoulder formed by projection 119 of the trigger button 112, thus no longer preventing distal displacement of the trigger button 112. In this first anchor deployment operative orientation, the trigger button 112 can be displaced both in proximal and in distal directions.

It is seen in FIG. 2 that in this first anchor deployment operative orientation, the first anchor 170 is optionally disposed distally with respect to the distal sharpened end 154 of the hollow needle 150, whereas the proximal end 172 of the first anchor 170 optionally abuts the distally facing end surface 159 of the pusher 130. The first anchor 170 is now deployed out of the interior volume 158 of the hollow needle 150 and within the tissue of the patient. The second anchor 180 is proximally spaced apart from the first anchor 170 in this first anchor deployment operative orientation and is still threaded over the outer surface of the pusher 130. The proximal end 184 of the second anchor 180 is optionally more distally spaced apart from distally facing surface 162 of the anchor stopper 160 than in the initial operative orientation.

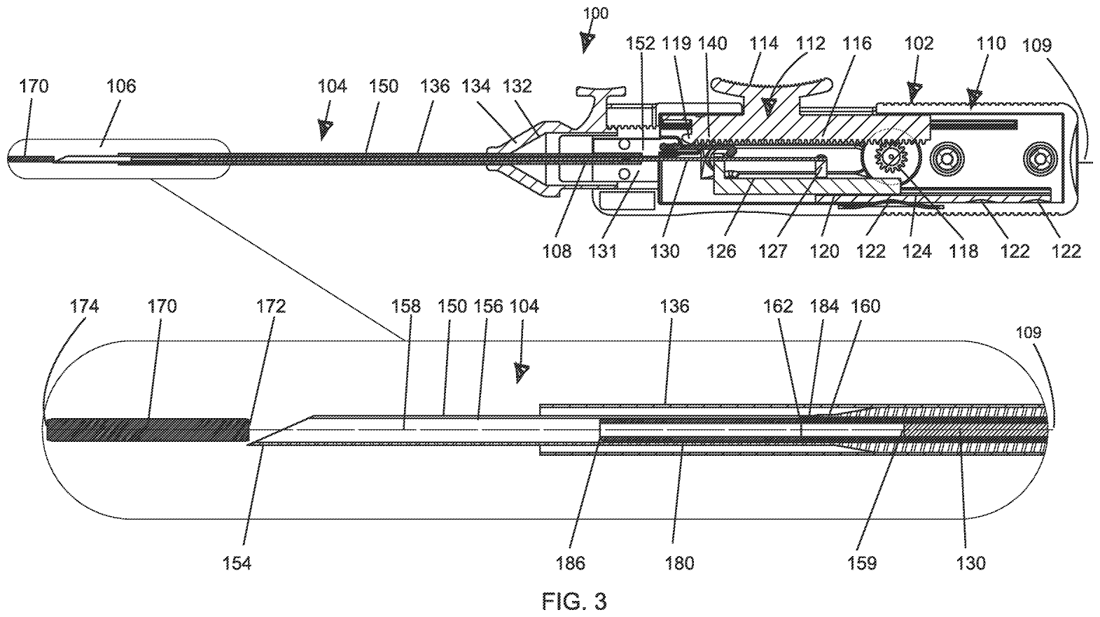

Reference is now made to FIG. 3, which is a simplified sectional view illustration of the multiple anchor delivery system 100 and an enlargement view of a distal end thereof, shown in a retraction operative orientation.

It is seen in FIG. 3 that in this retraction operative orientation, trigger button 112 is disposed in a distal position. In this position, the rack 120 is disposed in a proximal position, in which the leaf spring 124 is seated within yet another one of the indents 122 formed in rack 120.

The user displaced the trigger button 112 distally, thereby elongated toothed portion 116 of the trigger button 112 activated pinion 118, which in turn displaced the rack 120 proximally due to the interaction between the rack 120 and the pinion 118. Since the pusher 130 is connected to rack 120, the pusher 130 was thereby also retracted proximally.

During displacement of the rack 120, the leaf spring 124 is being positioned within a subsequent one of the indents 122, thereby potentially providing tactile indication to the user, indicating that the trigger button 112 is now positioned in the distal position. Alternatively, in some embodiments, proximal movement of the rack 120 from the position in FIG. 2 to the position in FIG. 3 results in the leaf spring being repositioned from being within a rightmost indent 122 (FIG. 2) to being within a leftmost indent 122 (FIG. 3).

It is a further particular feature of some embodiments of the present invention that in this retraction operative orientation, the safety latch 140 remains disengaged from the shoulder formed by projection 119 of the trigger button 112, thus no longer preventing distal displacement of the trigger button 112.

It is seen in FIG. 3 that in this retraction operative orientation, the first anchor 170 remains deployed outside of the hollow needle 150.

It is a particular feature of some embodiments of the present invention that during proximal retraction of the pusher 130, the second anchor 180 is released from the pusher 130 and is not mounted over the pusher 130 anymore, rather the proximal end 184 of the second anchor 180 abuts and is supported against distally facing surface 162 of the anchor stopper 160 in this retraction operative orientation, thus preventing proximal displacement of the second anchor 180.

It is seen in FIG. 3 that the distally facing end surface 159 of the pusher 130 is now proximally spaced from the proximal end 184 of the second anchor 180.

In this retraction operative orientation, the pusher 130 is ready for deploying the second anchor 180 in a manner similar to the deployment of the first anchor 170.

It is noted that an additional element may be formed on the distal end of pusher 130, such as, for example, a hook, to ascertain release of the second anchor 180 off the outer surface of the pusher 130 and loading thereof for deployment into the tissue of the patient.

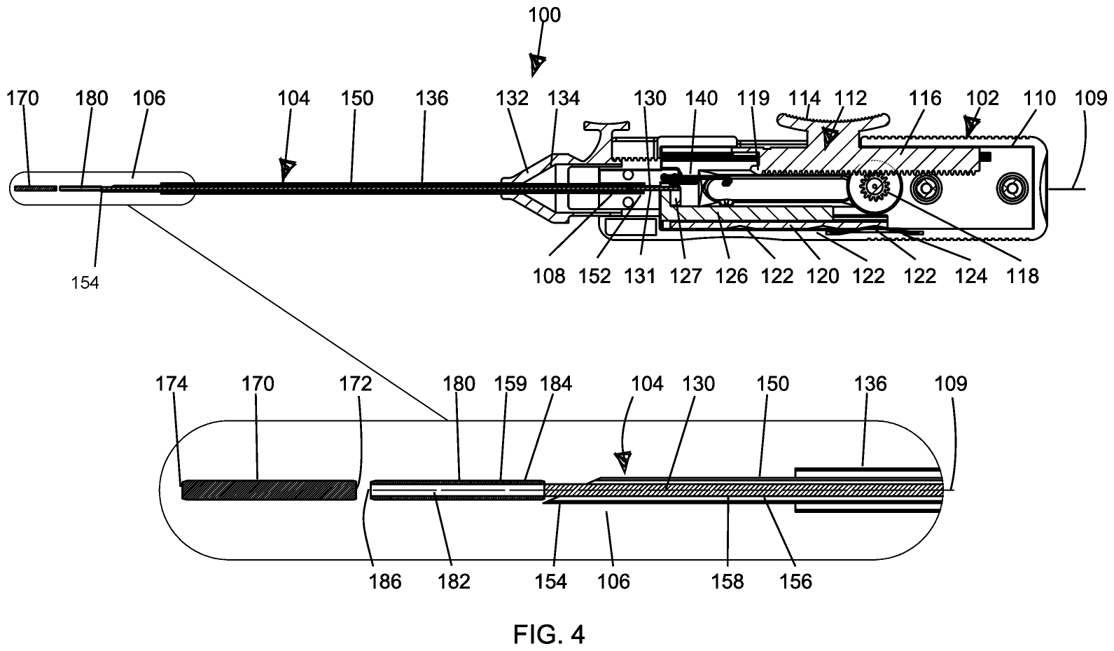

Reference is now made to FIG. 4, which is a simplified sectional view illustration of the multiple anchor delivery system 100 and an enlargement view of a distal end thereof, shown in a second anchor deployment operative orientation.

It is seen in FIG. 4 that in this second anchor deployment operative orientation, trigger button 112 is disposed in a proximal position again, similar to the position shown in FIG. 2. In this position, the rack 120 is disposed in a distal position, in which the leaf spring 124 is seated within another one of the indents 122 formed in rack 120.

The user displaced the trigger button 112 proximally, thereby elongated toothed portion 116 of the trigger button 112 activated pinion 118, which in turn displaced the rack 120 distally due to the interaction between the rack 120 and the pinion 118. Since the pusher 130 is connected to rack 120, the pusher 130 was thereby also displaced distally.

During displacement of the rack 120, the leaf spring 124 is being positioned within a subsequent one of the indents 122, thereby potentially providing tactile indication to the user, indicating that the trigger button 112 is now positioned in the proximal position. Alternatively, in some embodiments, proximal movement of the rack 120 from the position in FIG. 3 to the position in FIG. 4 results in the leaf spring being repositioned from being within a leftmost indent 122 (FIG. 3) to being within a rightmost indent 122 (FIG. 4).

The safety latch 140 remains disengaged from the shoulder formed by projection 119 of the trigger button 112, thus permitting displacement of the trigger button 112 in both proximal and distal directions.

It is seen in FIG. 4 that in this second anchor deployment operative orientation, the second anchor 180 is pushed distally due to distal displacement of the pusher 130 and due to engagement of distally facing end surface 159 of pusher 130 and the proximal end 184 of the second anchor 180. The second anchor 180 is now disposed distally with respect to the distal sharpened end 154 of the hollow needle 150 and is now deployed out of the interior volume 158 of the hollow needle 150 and within the tissue of the patient.

It is a particular feature of some embodiments of the present invention that the method of delivering multiple anchors into the tissue of the patient optionally includes delivery of the system 100 into the desired surgical location, displacement of a trigger button 112 in a first direction to deploy the first anchor 170, thereafter displacement of the trigger button 112 in a second direction, which is opposite to the first direction, to load the second anchor 180; and thereafter another displacement of the trigger button 112 in the first direction to deploy the second anchor 180. In some embodiments, the first direction is a proximal direction and the second direction is a distal direction.

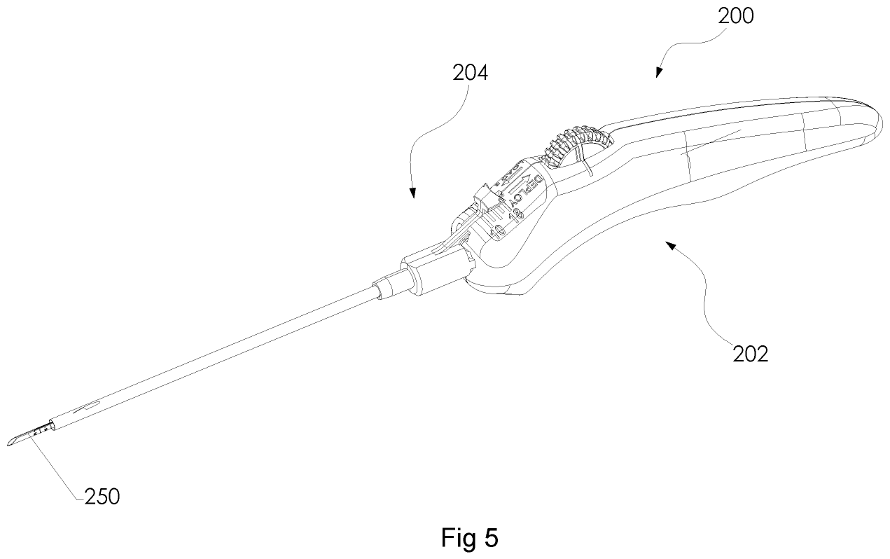

It is noted that the trigger button 112 described with reference to FIGS. 1-4 is displaced linearly, however another type of trigger button may be used, such that can be alternatively rotated in order to advance or retract the pusher and thereby optionally affect deployment of anchors. An exemplary rotary trigger is discussed further herein with reference to FIGS. 5-23E.

It is appreciated that the system 100 provides an indication to the user that the second anchor 180 is loaded and ready to be deployed.

It is appreciated that alternatively the system 100 can deploy the first anchor 100 and thereafter during retraction of the pusher, the second anchor 180 that was initially disposed out of the interior volume 158 of the needle 150 is loaded onto the pusher 130 and is now ready for deployment. For example, needle 250 may include a recess on its inner surface, and second anchor 280 may be pushed into the recess before or after pusher element 230 is retracted to its most proximal position.

Further alternatively, both first anchor 170 and second anchor 180 may be loaded together in parallel, and each of the anchors is optionally covered by a resilient cover. Both anchors are loaded together into a single hollow needle 150 and a driving assembly having a first pusher that deploys the first anchor 170 and a second pusher that deploys the second anchor 180.

It is noted that the system 100 is optionally configured to be disposable. After use, the device may be disposed of in a disposal container approved by the local authority.

It is noted that the trigger button 112 described with reference to FIGS. 1-4 is displaced linearly. However another type of actuator may be used, such as, for example, a roller 212, that may be rotated in order to advance or retract the pusher and thereby optionally affect deployment of anchors. An exemplary rotary trigger is discussed further herein with reference to FIGS. 5-23E.

### Some Exemplary Embodiments

With reference to FIG. 5 there is shown an exemplary multiple anchor delivery system 200, in accordance with some embodiments of the invention. The system includes a multiple anchor delivery device 204 including a handle 202, a cannula 236, and a sheath 250.

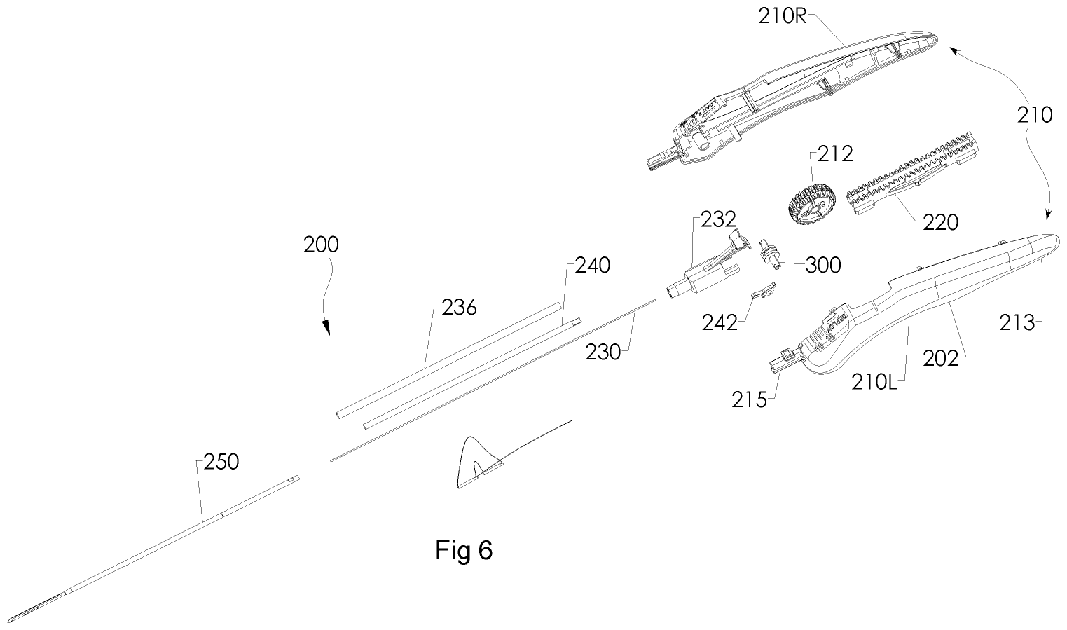

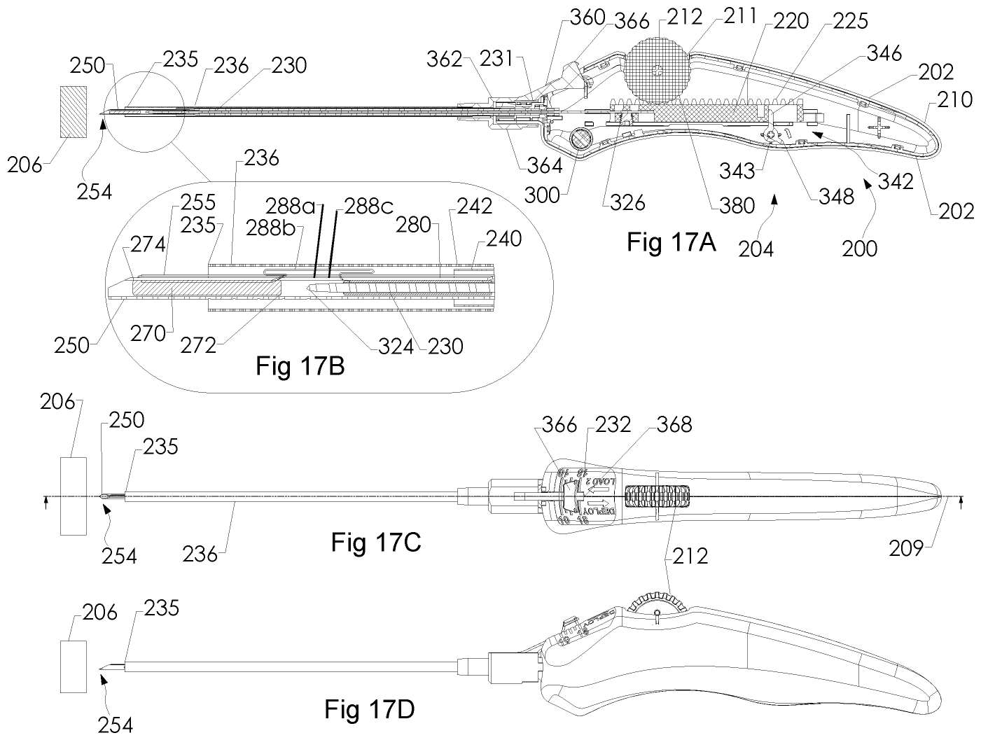

With additional reference to FIG. 6, there is shown an exploded view of system 200 shown in FIG. 5, wherein some aspects of some components of the system may be more clearly shown. The handle 202 is shown having a housing 210 with a proximal end 213 and a distal end 215, the housing 210 including a right housing portion 210R and a left housing portion 210L. Components to be at least partially housed in the housing 210 include a roller 212, a rack 220, a bobbin 300, and a locking element 242. Also shown are a sheath 250, a pusher element 230, an optional suture holder 240, a cannula 236, and a delimiter 232, all of which are discussed further herein with regard to FIGS. 17A-22C.

In accordance with some embodiments, some of the components shown in FIG. 6 may be omitted or replaced by other components. For example, suture holder 240 may be omitted, and/or roller 212 and/or the rack 220 may be replaced with other components that are coupled together to drive the pusher element distally and proximally through the sheath.

Optionally, in some embodiments, the bobbin 300 may be replaced by another mechanism for storing the length of suture that extends from the first anchor into the handle. Optionally, there is no mechanism for storing the length of suture, and the length of suture may be stored inside the housing 210 or at least partly extends outside the housing.

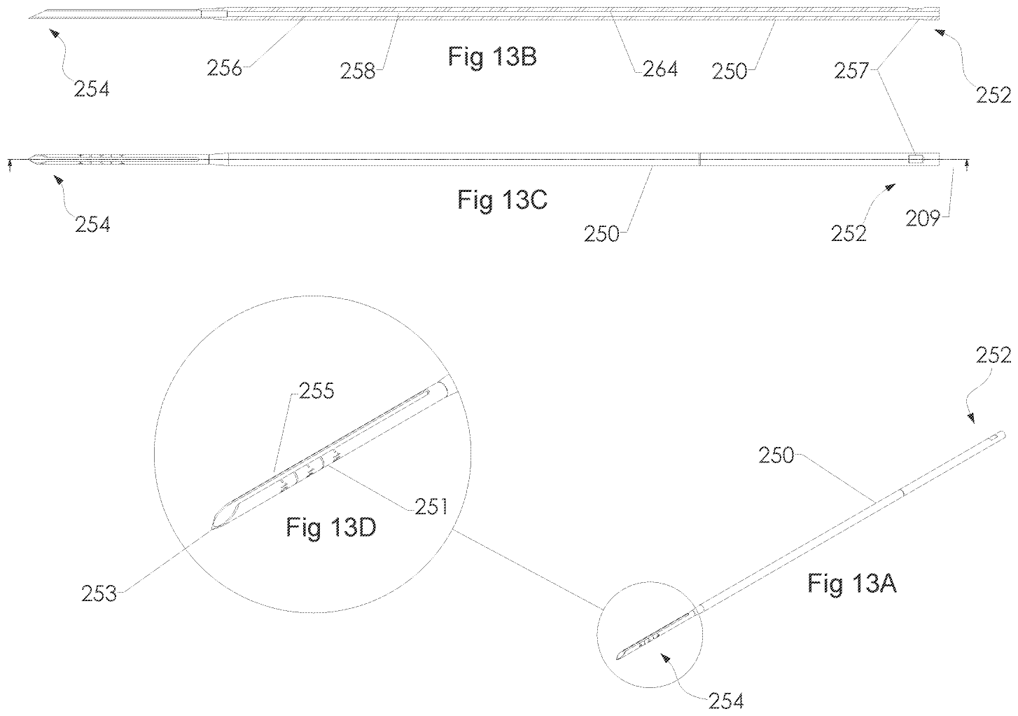

FIGS. 13A-D illustrate an exemplary sheath 250 in accordance with embodiments of the invention. It will be appreciated by persons skilled in the art that “sheath” may be used as a general term to indicate any elongated component having a hollow lumen, for example, a lumen with a circular or ovate cross-section. The term “needle” is generally used to describe a component that is inserted into the human body. A “needle” may, therefore include a “sheath” or other tube and optionally includes a sharpened end, for example, as discussed herein.

In the embodiment shown, sheath 250 may be configured as a hollow needle having a circular cross-section. Needle 250 has a proximal end 252 and a distal end 254 and may be fabricated of, for example stainless steel of grade SS304, although other materials are possible. Needle 250 may have a length in the range of, for example, 176.3-176.7 mm; an outer diameter in the range of, for example, 1.8-2.55 mm; and an inner diameter in the range of, for example, 1.15-1.5 mm.

The distal end 254 is optionally provided with a sharpened tip 253 configured to penetrate tissue, such as, for example, a hypodermic needle. A portion of needle 250, adjacent needle distal end 254, may have a lateral opening formed therein, such as, for example, slot 155, optionally opposite the side having the sharpened tip 253. Slot may have a length in the range of, for example, 29-30 mm and a width in the range of, for example, 0.8-0.9 mm.

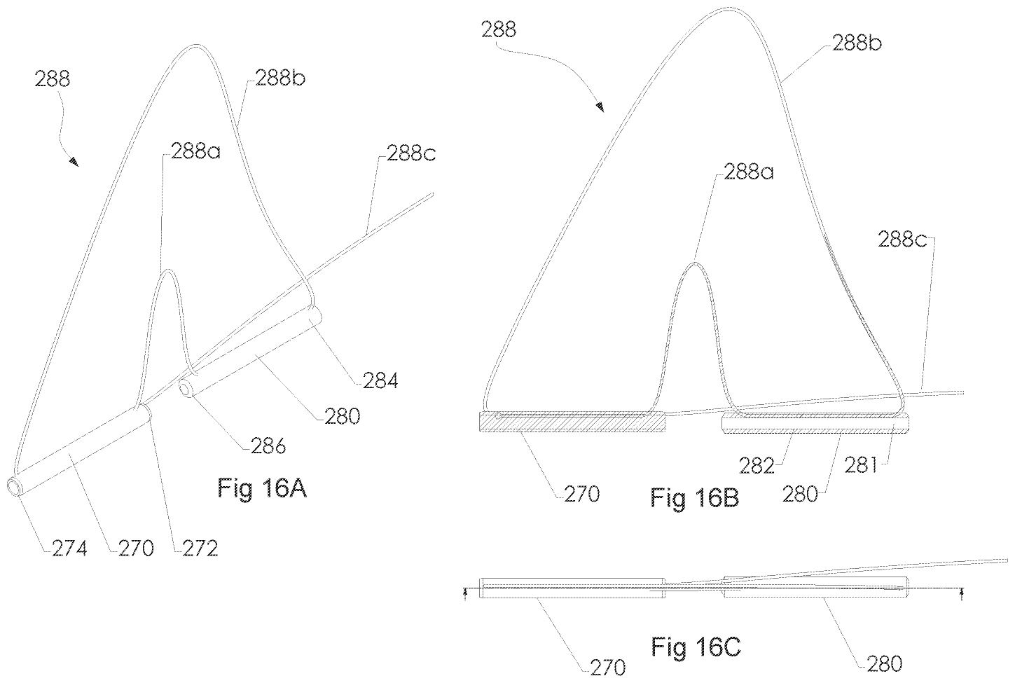

Slot 255 is optionally large enough to accommodate the passage therethrough of suture loops 288a-b and suture free end 288c, for example, as discussed further herein, for example with regard to FIGS. 16A-C. Optionally, the distal end 254 of needle 250 is provided with laser markings, for example, laser markings of a type known in the art, which may be used as a reference to indicate a depth of insertion of the needle 250 into a tissue. Near the needle proximal end, there may be provided a pair of indentations 257. These and/or other optional features of the needle will be discussed further herein.

While, in the embodiment shown, needle 250 is shown as having a straight configuration, it is to be understood that, optionally and alternately, the needle distal end 254 may be curved, as known in the art. Needle 250 may have a curvature having a radius of curvature in the range of, for example, 99-100 mm; for a length in the range of, for example, 20-22 mm; and a height in the range of, for example, 2-3 mm.

Optionally, the needle may be bendable, for example, which may prevent damage to tissue. Optionally, the needle may have more than one curve and/or curve in more than one plane. It will be appreciated by person skilled in the art that if the needle is curved the pusher element is optionally thick enough and flexible enough to avoid buckling as it is displaced along the curved needle.

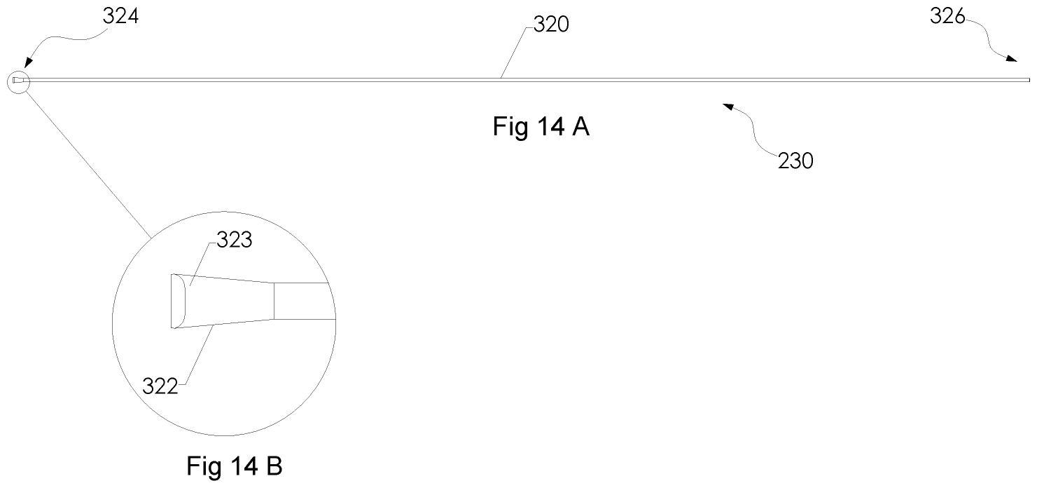

FIGS. 14A-B show an exemplary pusher element 230 in accordance with some embodiments of the invention. Pusher element 230 may be configured generally as an elongated rod 320 having a proximal end 324 and a distal end 326. Pusher element 230 may have a length in the range of, for example, 197-197.2 mm and an outer diameter in the range of, for example, 1.1-1.2 mm. Pusher element 230 is optionally a solid component, and is optionally made of stainless steel, for example, of grade SS302 However, other configurations and other materials for fabricating pusher element 230 are conceivable. Optionally, pusher element 230 has a circular cross-section, although other configurations are conceivable, such as, for example, elliptical.

A pusher tip 322 may be provided at the pusher proximal end 324, the tip 322 optionally being compressible. Optionally, pusher element 230 has a widened portion 323 at tip 322. Optionally, widened portion 323 may be compressible. The pusher element is optionally flexible enough to be resistant to buckling and to be able to transfer a force required to overcome friction between each of the anchors 270/280 and an inner surface 256 of the needle 250. Optionally, the needle inner surface 256 is coated to reduce such friction.

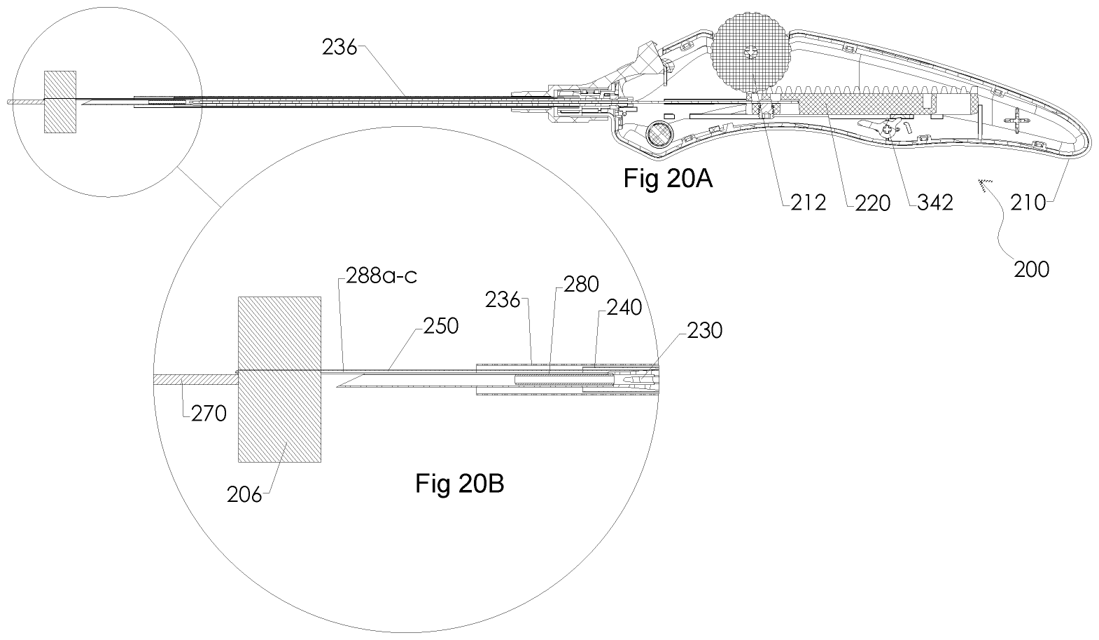

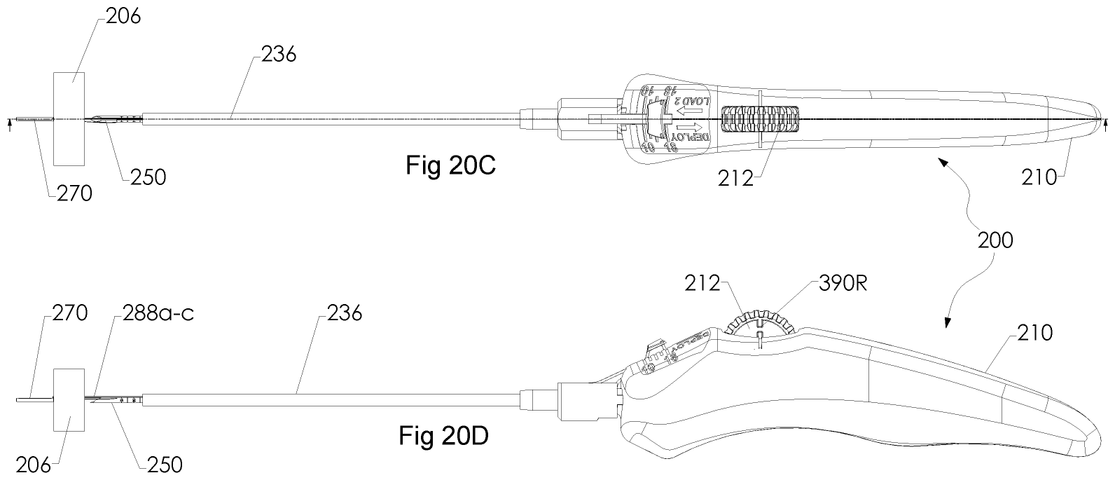

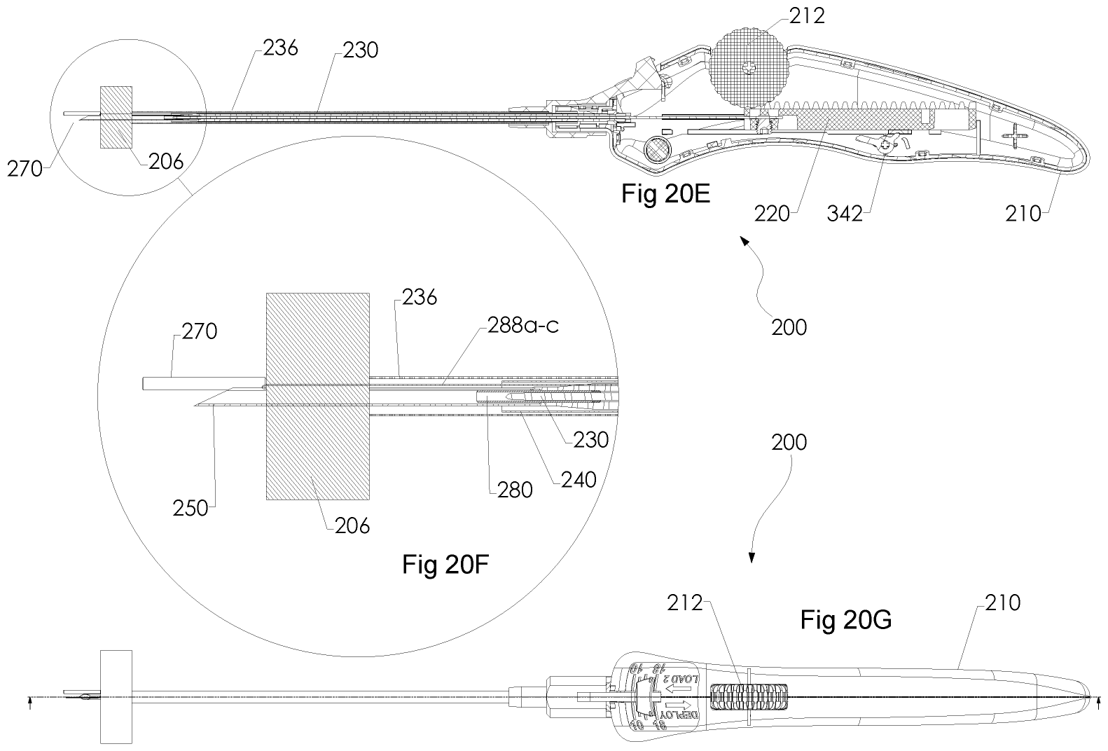

According to some embodiments, the pusher element may be provided with an indentation or notch for retaining therein a second anchor. Optionally, the pusher element distal end and/or the second anchor may be compressible. When the pusher element is retracted, for example, as discussed herein with regard to FIG. 20A, the second anchor is prevented from moving proximally by a barrier positioned within the passageway such as, for example, a narrowing of the passageway and/or any structural component that interferes with proximal movement of the second anchor in the passageway. As the pusher element is retracted, a portion of the pusher element distal to the indentation slides over the second anchor so that the second anchor may be released from the pusher element indentation, the pusher element distal end being proximal to the second anchor proximal end. Then the compressed pusher element distal end and/or second anchor may decompress such that the pusher element is wide enough to push the second anchor out of the needle, for example, as discussed below.

According to some embodiments, the sheath may be provided with a recess or notch for the second anchor, and the pusher element may slide past the second anchor when deploying the first anchor. When the pusher element is retracted it may release the second anchor from the needle recess, by no longer blocking it from decompressing into the needle lumen, thereby potentially allowing the second anchor to be positioned for subsequent deployment.

FIGS. 16A-C show an exemplary first anchor 270, having proximal end 272 and distal 274, and an exemplary second anchor 280, having a proximal end 284 and a distal end 286. In the embodiment shown the first anchor 270 may be generally cylindrical and may be solid, while the second anchor 280 may be generally cylindrical and may be hollow, for example, having an interior 281 bounded by an inner surface 282 of second anchor 280. However, it will be appreciated by persons skilled in the art that, optionally, the anchors may have other configurations, for example, as discussed herein.

Each of first anchor 270 and second 280 may be fabricated from 8-16 braided or woven strands of USP 4-0 or 2-0 Fiber Wire™, although other materials and/or sizes may be usable such as, for example, ultra-high molecular weight polyethylene (UHMWP), polyester polypropylene, or silicone elastomer coating, optionally dyed using D&C Blue No. 6, D&C Green No. 6, and/or Logwood Black dyes, as known in the art.

Optionally, either or both of anchors 270 and 280 may include a silicone elastomer coating. Each of anchors 270 and 280 has a length in the range of, for example, 11-13 mm; and an outer diameter in the range of, for example, 1.2-1.4 mm. Second anchor 280 may have an inner diameter in the range of, for example, 0.65-0.86 mm. A length 288 of suture material may extend through the anchors, optionally having a small portion 288a forming a small loop between first anchor proximal end 272 and second anchor distal end 286, a large portion 288b forming a large loop between the first anchor distal end 274 and the second anchor proximal end 284, and a free end 388c extending proximally from the first anchor proximal end. The length 288 of suture material may be formed of, for example, 2-0 Fiber Wire™, although other materials may optionally be used. Optionally, the length of the small portion 288a of suture material is 50 mm, the length of the large portion 288b of suture material is 220 mm, and the length of the suture free end 288c is 300 mm, although other lengths may be used.

One or both of first anchor 270 and second anchor 280 are optionally soft and/or flexible enough so as not to cause damage to tissue or vessels in the vicinity of the implant. Optionally, either or both of first anchor 270 and second anchor 280 may be rigid. Optionally, either or both of first anchor 270 and second anchor 280 may be bio-absorbable, optionally maintaining mechanical strength of the anchor or anchors until tissue 206 is healed such as, for example, 1-3 months.

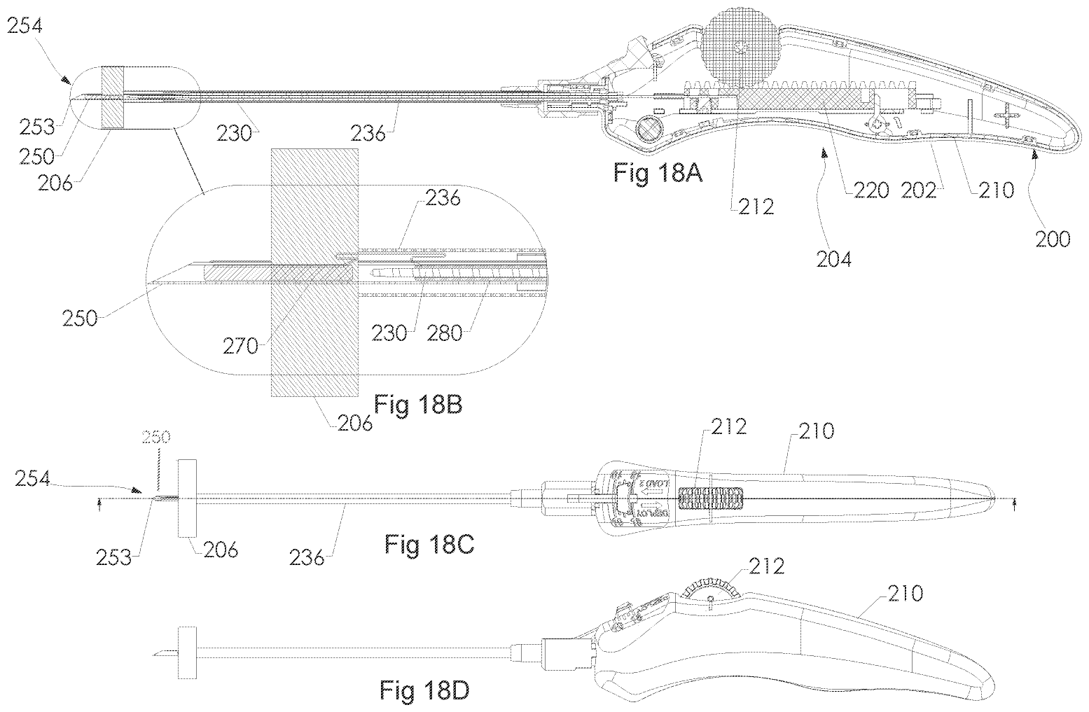

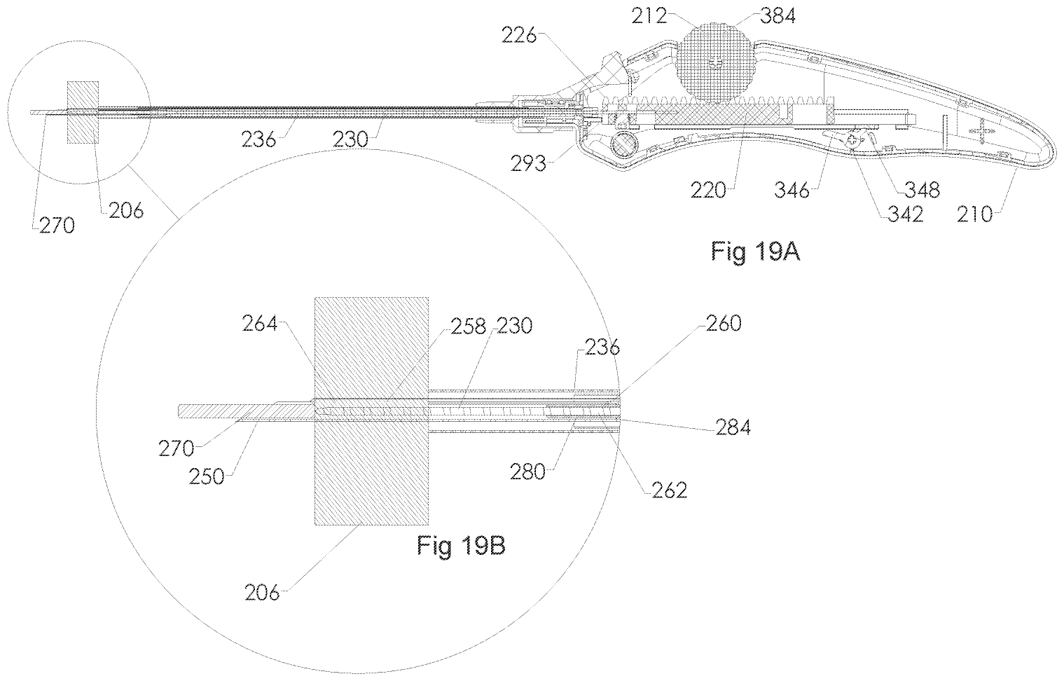

For example, as discussed herein, in accordance with some embodiments, optionally, the first anchor may be a solid anchor optionally having a blind bore at a proximal end thereof, into which a pusher element distal end may be inserted prior to deployment of the first anchor. In this embodiment, the pusher element distal end may not contact the first anchor proximal end, as in the embodiment shown in FIG. 18A, but instead the pusher element distal end may contact the end surface of the blind bore within the first anchor.

In accordance with a further alternative embodiment, the first anchor 270 may be hollow, and the pusher element may be disposed proximal relative to the first anchor, the pusher element configured to contact the first anchor proximal end, to deploy the firs anchor. Optionally or additionally, the pusher element may be disposed partly within the interior of the first hollow anchor, the pusher element having a distal end configured to engage the inner surface of the first anchor, thereby potentially deploying the first anchor when the pusher element is moved distally.

In some embodiments of the invention, second anchor 188 may optionally be replaced with a solid anchor which is optionally compressible, for example, as discussed herein with regard to FIGS. 16A-C.

In some embodiments, the anchors are provided with suture portions which allow tightening of the anchors against the tissue after deployment of the anchors. Optionally, suture material is threaded at least partly through the interior of second anchor 280. Optionally, suture material may be threaded at least partly through the material of first anchor 270 and/or the material of the second anchor 280.

Optionally, each of a first anchor 270 and a second anchor 280 may have a collar or channel (not shown), extending alongside an outer surface of the anchor, the channel configured for retaining therein a portion of suture material 288. The anchors 270 and 280 may be positioned within the needle 250 such that the suture portions 288a-b and suture free end 288c and/or the channels protrude out of the needle slot 255.

It should be noted that, depending on the shape of the bore through the hollow anchor 280 and the shape of an opening into the bore of the second anchor, the second anchor 280 may be flexible enough and the distal end 324 of pusher element 230 may be narrow enough so that the pusher element may pass through the interior 281 of the second anchor 280. In addition, distal end 324 of the pusher element 230 may be wide enough so that, after retraction out of the second anchor 280, the pusher element will push the second anchor distally. This is discussed further below.

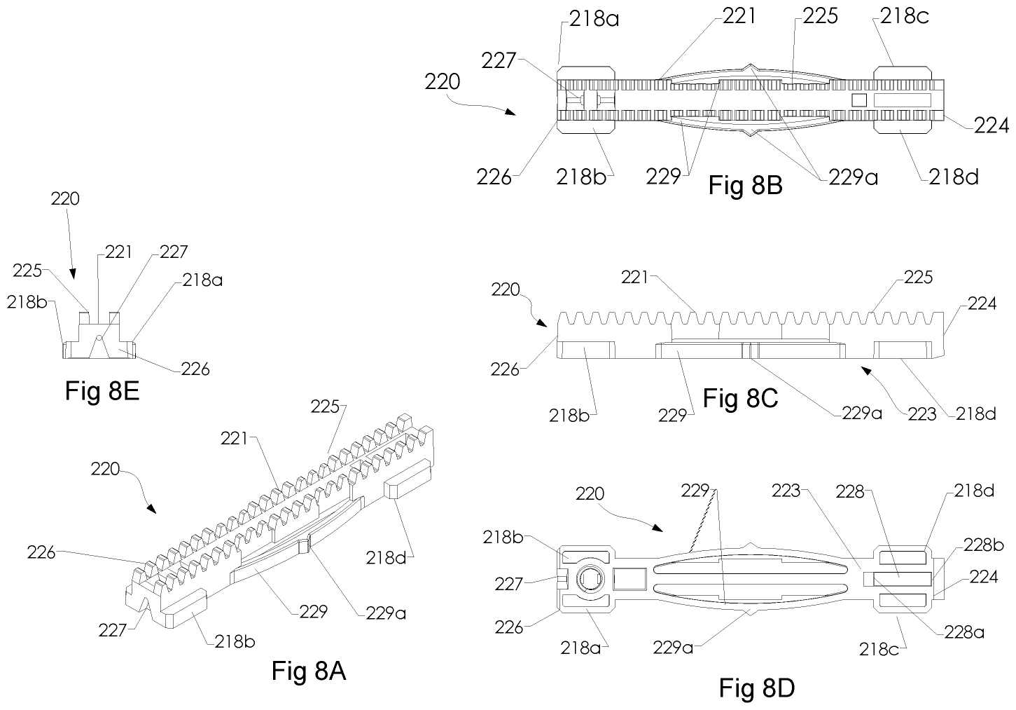

Referring to FIGS. 8A-E there are shown a rack 220, in accordance with some embodiments of the invention. For example, as discussed further with regard to operation of the system 200, rack 220 may be coupled to the roller for displacing the pusher element 230 through the needle passageway 264. Rack 220 has a proximal end 224 and a distal end 226. The upper surface 221 of rack 220 may be provided with a plurality of evenly spaced teeth 225 extending along the length of the rack. While, in the embodiment shown, rack 220 is provided with two rows of teeth, it will be appreciated by persons skilled in the art that, optionally, rack 220 may have a single row of teeth extending along at least a portion of the length thereof.

According to some embodiments, a lower surface 223 of rack 220 may be provided with a recess 228 having distal and proximal surfaces 228a-b, near the rack proximal end 224. The rack 220 may also be provided with flexible strips 229 which appear as curved portions protruding from the sides of the rack. The middle of each strip 229 has a v-shaped projection 229a extending away from the rack 220. The rack 220 may include a plurality of support portions 218a-d extending out of the sides of the rack 220, where support portions 218a-b are positioned at the rack distal end and support portions 218c-d are positioned near the rack proximal end. One or more of these elements may be described in more detail herein.

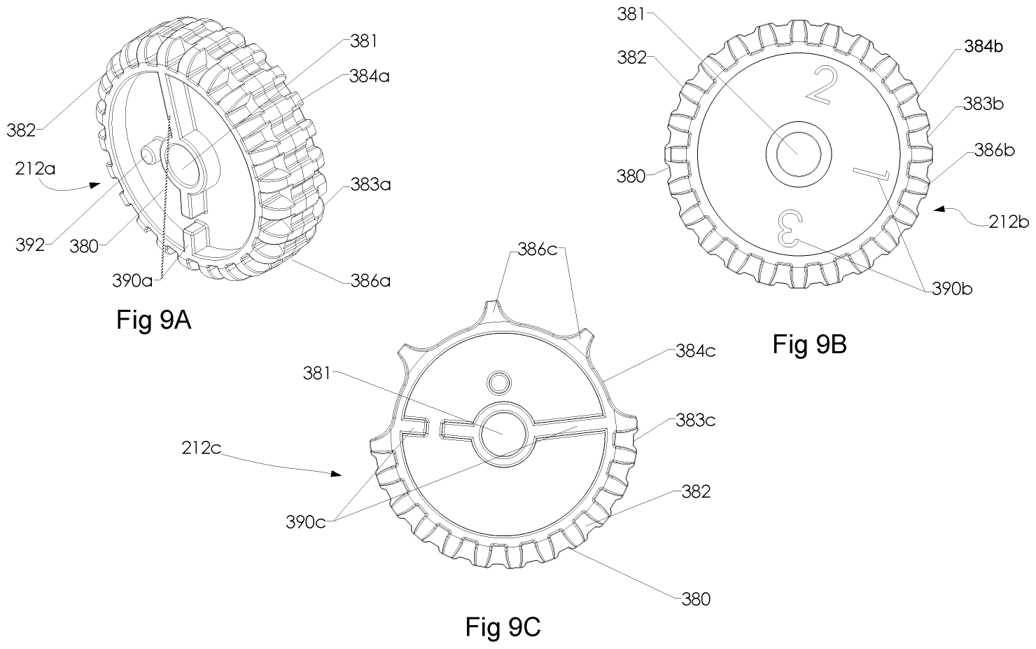

With reference to FIGS. 9A-C there are shown a plurality of exemplary rollers 212a-c, in accordance with some embodiments of the invention. A roller 212 is a hand-movable component may be coupled to the rack 220 so that movement of the roller actuates the rack to be displaced proximally or distally within the housing, for example, as discussed further with regard to operation of the device. Each roller 212a-c may have a generally circular configuration with a circular bore 381 disposed at the center thereof. Each roller 212a-c includes an operative portion 380 optionally on approximately half of a respective outer periphery 383a-c thereof, the operative portion 380 defined by a plurality of optionally evenly spaced teeth 382. The rollers 212a-c may also include a respective gripping portion 384a-c on approximately the remaining half of the outer periphery of the roller. In some embodiments (FIGS. 9A-B) the gripping portion 384a-b may include a plurality of evenly spaced teeth 386a-b similar to those of the operative portion 382. However, the gripping portion 384c may, alternatively, include fewer teeth such as, for example, only three teeth 386c (FIG. 9C), which are optionally evenly spaced along the gripping portion 384. It should be noted that the teeth 382 of operating portion are optionally configured so as to be engageable with corresponding teeth 225 of rack 220 (FIG. 4A).

A roller 212c may optionally be provided with a peg 392 projecting out of the surface 391 of the roller 212a-c, which may be used for positioning the roller within the housing.

A roller 212a-c may optionally be provided with position markings 390a-c for indicating to a user the rotational position of the roller. For example, as shown in FIG. 9B, roller 212b may have position markings “1,” “2,” and “3.” Optionally, a roller 212a-c may be provided with markings 390D-N-R, for example, to indicate respective deployment, neutral, and retracted positions, or with any other markings that indicate to the user a position of the roller. Optionally, an electronic circuit may be provided to indicate to the user the current position of the roller 212.

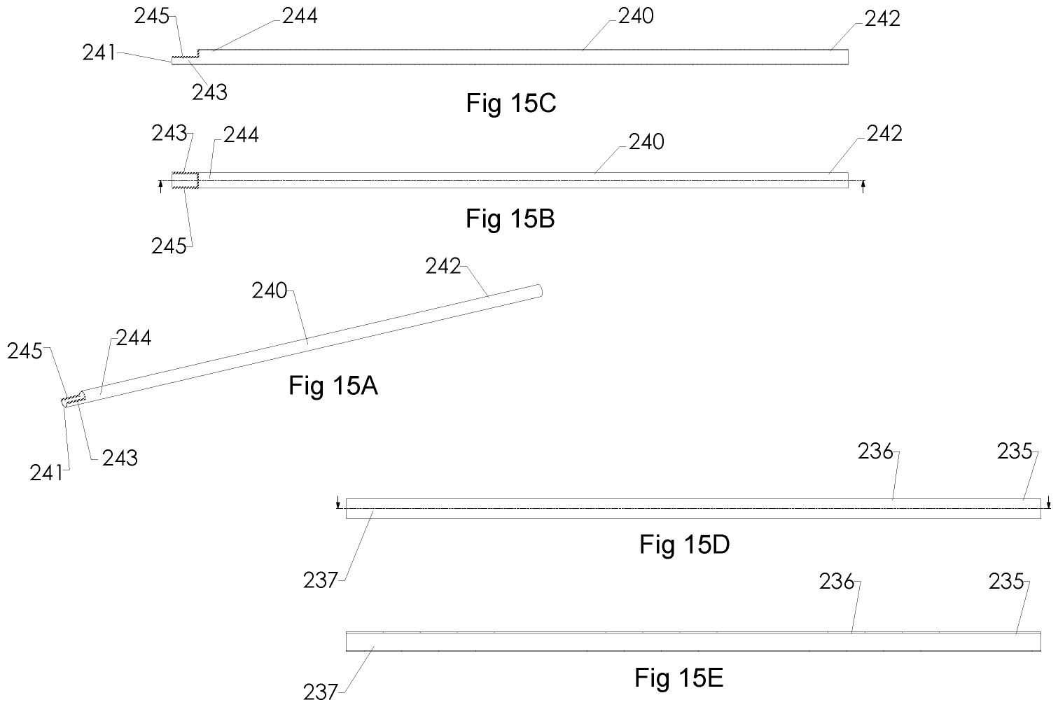

With reference to FIGS. 15D-E there is shown an exemplary cannula 236, in accordance with some embodiments of the invention. Cannula 236 may be fabricated from PTFE and has a length in the range of, for example, 135-137 mm; an outer diameter in the range of, for example, 3.5-3.6 mm; and an inner diameter in the range of, for example, 3.3-3.4 mm.

Cannula 236 is positioned over the needle and over the suture holder, if it is present. The cannula distal end 235 may be moved relative to the needle distal end 254, to adjust the length of the needle that extends out of the cannula. This is discussed further herein with regard to delimiter 360. It should be noted that the inner diameter of the cannula 236 must be larger than the outer diameter of the needle 250, such that the needle 250 may be inserted into the cannula 236, optionally with a suture holder 240 in between the cannula inner surface and the needle outer surface.

FIGS. 15A-C show an exemplary suture holder 240, in accordance with some embodiments of the invention. Suture holder 240 is optionally utilized to retain suture loops 288a-b adjacent needle 250, and optionally to prevent tangling of the suture material as the anchors are displaced within the needle 250. After anchors 270 and 280 (FIGS. 16A-C) have been inserted into the needle 250, with the suture loops 288a-b extending through slot 255, a suture holder 240 may be positioned over the needle for retaining the sutures therein. Optionally, suture holder 240 may be omitted from the system 200.

Suture holder 240 is configured as a generally cylindrical tube having a distal end 242 and a proximal end 244. Suture holder 240 may be fabricated of PTFE and has a length in the range of, for example, 130-132 mm; an inner diameter in the range of, for example, 2.6-.2.8 mm; and an outer diameter in the range of, for example, 3.0-3.2 mm; with a cutaway portion extending 5-6 mm from the suture holder proximal end 244.

At its proximal end 244, suture holder has a cutaway portion such that the proximal end is provided with a semicircular arm 243 having a horizontal edge 245. Edge 245 intersects a vertical edge 241 at a right angle. The suture holder 240 will be discussed further herein with regard to assembly of components of the system 200.

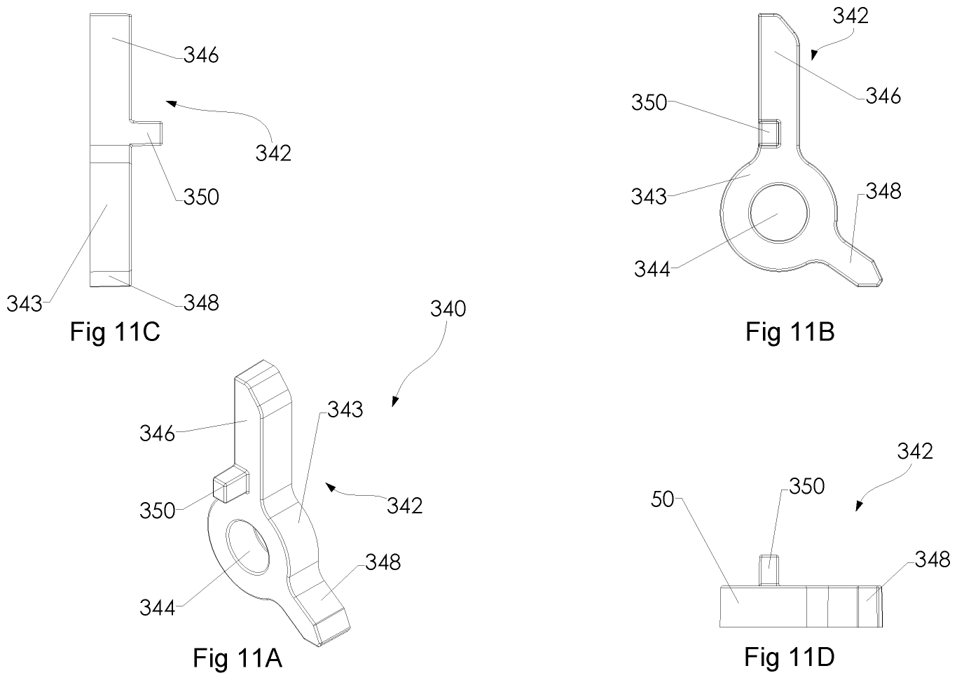

It should be noted that the inner diameter of the suture holder 240 must be larger than the outer diameter of the needle 250, such that the needle 250 may be inserted into the cannula 236. In addition, the inner diameter of the suture holder should be large enough so that there is enough room in between the inner surface of the suture holder and the outer surface of the needle for the presence of suture loops 288a-b. With reference to FIGS. 11A-D there is shown an exemplary locking element 342 which may be provided as part of an optional locking mechanism 340, in accordance with embodiments of the invention. This is discussed further herein. The locking element 342 may have a generally circular body 343 having a generally circular opening 344 therethrough. A locking bar 346 configured as a long arm and a short arm 348 may extend from the circular body 343. A peg 350 may project out of the surface of the locking bar 346, the peg having been formed during manufacture of the locking element 324. Locking element 342 will be discussed further herein with regard to FIGS. 17A-22C.

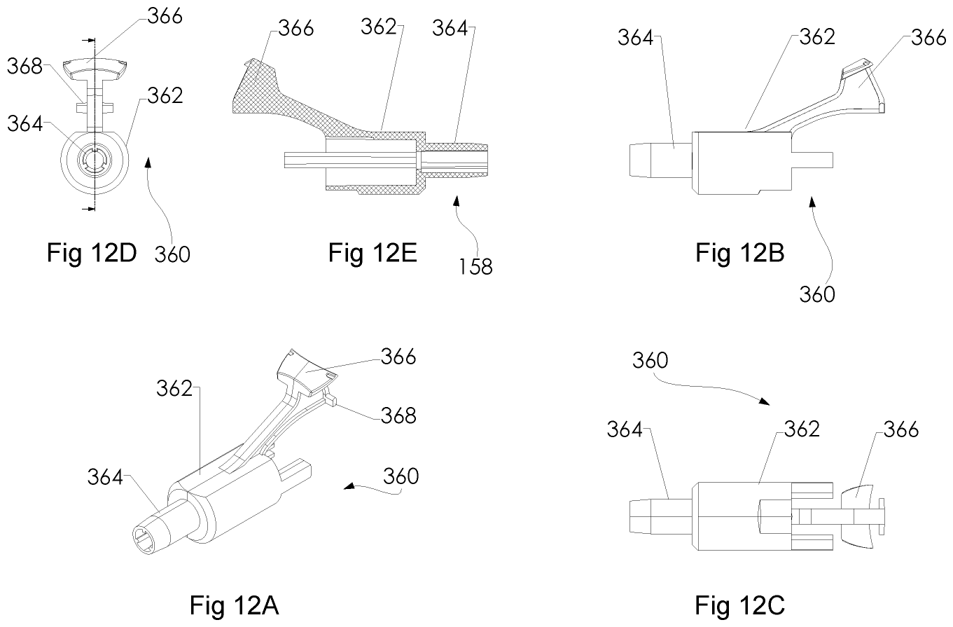

FIGS. 12A-E illustrate an exemplary needle length delimiter 360, in accordance with embodiments of the invention. Delimiter 360 may include a hub 362 and a circular collar 364. A pair of arms 361 extends from the hub 362, away from the collar 364. A slider 366 may extend proximally from the hub 362, the slider having an indicator peg 368 thereon. The delimiter 360 will be discussed further herein with regard to assembly of system 200. The delimiter 360 may be fabricated from any suitable material such as, for example, polytetrafluoroethylene (PTFE). Optionally, delimiter 360 may be omitted from system 200.

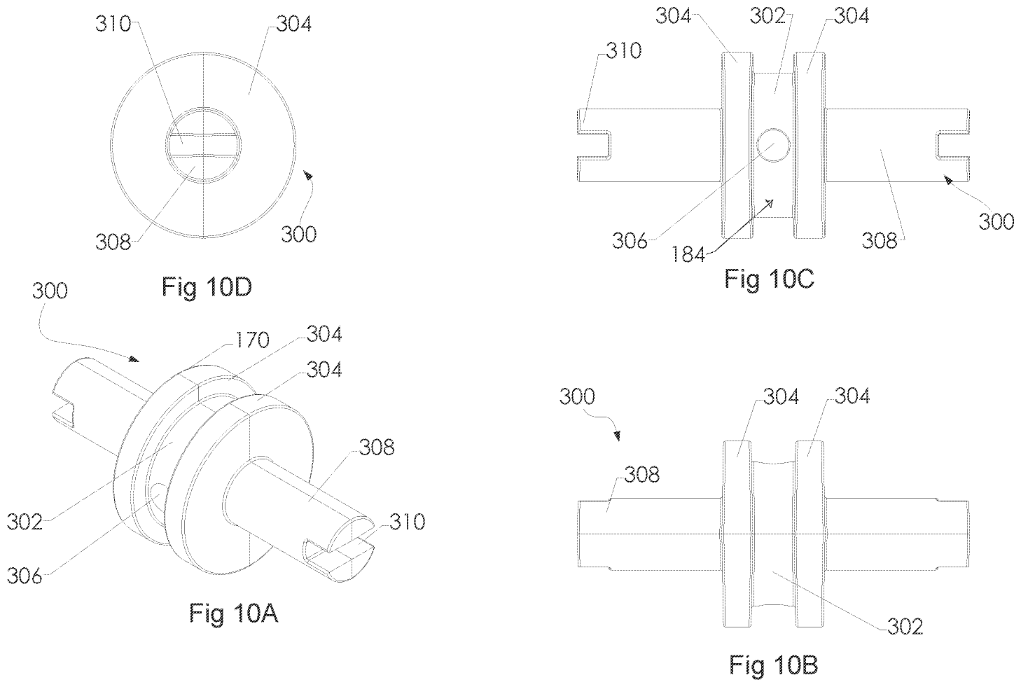

FIGS. 10A-D illustrate an exemplary bobbin 300 according to some embodiments of the invention. Bobbin 300 optionally includes a spool 302 bounded on either side by flanges 304. A bore 306 may extend through spool 302. Spool 302 is provided with a rod 308 extending through the spool. The rod is optionally provided with grooves 310 at either end thereof. The bobbin will be discussed further herein with regard to assembly of system 200.

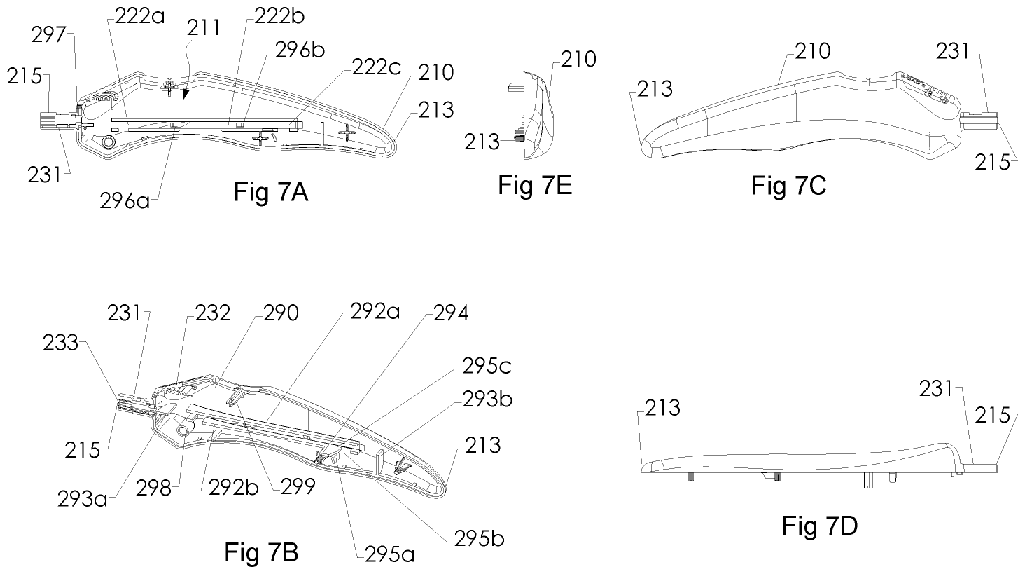

With further reference to FIG. 7A-E, there are shown further details of an exemplary right half portion 210R of a housing 210, in accordance with some embodiments of the invention. Housing 210 may be fabricated of acrylonitrile butadiene styrene (ABS), although other materials are possible.

The inner wall 290 of right housing portion 210R may be provided with a pair of parallel flanges, an upper flange 292a and a lower flange 292b, between which there may be situated projections 296a and 296b. A first recess 222a may be formed between flanges 292a and 292b, proximal to projection 296a; a second recess 222b may be formed between flanges 292a and 292b, between projections 296a and 296b; and a third recess 222c may be formed between flanges 292a and 292b, distal to projection 296b. Also on the inner wall 290 of right housing portion 210R there may be provided a roller mounting peg 299, locking element stops 295a and 295c, a bobbin holder 298, stops 293a-b, and a locking element peg 294. It may be noted that stop 295a is configured as a curved portion projecting out of housing inner wall 290, the curved portion having a small knob 295b at its upper end. Near the housing distal end 215 there may be provided clamping bars 297. These elements will be discussed further herein with regard to assembly and operation of system 200.

It will be appreciated by persons skilled in the art that, optionally, a left housing portion (not shown) may be formed similar to housing portion 210R, but with components in a laterally reversed configuration. The left and right housing portions 210L-R may be configured to be snap fitted together, or to be otherwise joined, for example, by being glued or screwed together, as known in the art, to form a housing 210.

The housing 210 may be fabricated from any suitable material such as, for example, plastic, optionally by injection molding. The housing 210 may be configured to partly contain a mechanism responsible for delivery of anchors through a tissue, for example, as discussed herein with regard to assembly and operation of system 200.

### Assembly of Components of the System

In accordance with some embodiments, system 200 may be assembled as follows: First the proximal end 326 of pusher element 230 may be inserted through the distal end 286 of hollow anchor 280. It may be noted that the inner surface 282 of the second anchor 280 may or may not engage an outer surface of the pusher element 230.

The pusher element proximal end 326 may then be inserted through the distal end 254 of a needle 250 until it extends out of the needle proximal end 252, and the hollow anchor may be positioned within the needle 250, optionally with suture loops 288a-b positioned outside the needle slot 255. Then the first anchor may be inserted into the needle distal end 286, optionally with the suture loops 288a-b positioned outside the needle slot 255, and the suture free end 288c optionally extending proximally within the needle. Optionally, the small and large suture loops 288a-b may be adjusted such that they have the desired measurements, for example, as discussed herein with regard to FIGS. 16A-C. Optionally, the suture free end 288c may be twisted around the large suture loop 288b, so that a knot may be formed after deployment of the anchors 270 and 280, for example, as discussed herein. It should be noted that, in this configuration, the first anchor 270 may be disposed distally relative to the pusher element distal end 324, and that the second anchor 280 may be disposed proximal to the proximal end 272 of the first anchor 270.

The pusher element proximal end 326 may then be inserted into bore 227 in rack 220 and attached thereto, for example, by a set screw, making sure that the needle is positioned with the slot on the same side as the teeth 225 of the rack 220.

Needle proximal end 252 may be inserted into the distal end 242 of a suture holder 240, and the suture holder may be passed over the needle, optionally until almost all of the needle slot 255 extends past the suture holder distal end 242. Optionally, approximately 2-3 mm of the needle slot 255 is covered by the suture holder 240.

A locking element 342 may be positioned relative to rack 220 such that the locking bar 346 is disposed within the recess 228 in rack 220. Then the rack and locking element are positioned within a half portion of a housing 210, e.g., right housing portion 210R, with rack support portions 218b and 218d disposed in recesses 222a and 222c, between housing flanges 292a and 292b; with small arm 348 on locking element 342 below stop 295a in housing 210, and with cannula positioned within housing hub 231.

In some embodiments, at this point the suture holder 240 may be adjusted so that the semicircular arm 243 is positioned inside the right portion of housing collar 231 in housing portion 210R, with the semicircular arm oriented such that it forms a complete circle together with the right portion of collar 231, and with vertical edge 241 of suture holder 240 abutting the edge 233 of housing collar 231. This ensures that the suture free end 288c passes through the circle formed by the semicircular arm 243 and the right portion of housing collar 231, thereby potentially preventing possible accidental closure of the housing portions 210L-R on the suture free end 288c.

The suture free end 288c may be optionally threaded through the bore 306 in bobbin 300 and the suture may be wound around bobbin spool 302, after which bobbin may be positioned in housing portion 210R, with bobbin rod 308 in holder 398 of right housing portion 210R. The portion of suture free end 288c that extends between the cannula proximal end 237 and the spool 302 should be positioned within the housing hub 231. The bobbin may be wound tighter, if desired, optionally by inserting a tool into groove 310 at the end of rod 308.

A roller, such as, for example, roller 212c may be positioned in the right housing portion 210R with opening 381 on the roller mounting peg 299 in right housing portion 210R, with the peg 392 outside the right housing portion 210R, the peg 392 aiding in positioning of the roller relative to the housing portion 210R. In this configuration, the roller operative portion 380 is optionally disposed inside the right housing portion 210R, and the roller gripping portion 384c is optionally disposed outside the right housing portion 210R.

The left housing portion 210L may then be snap fitted onto or otherwise attached to the right housing portion, to form a closed housing 210 with the rack 220, locking element 342, and bobbin 300 inside, and with the roller 212a, partially inside and partially outside of the housing 210. Finally, the needle distal end 254 and cannula distal end 235 may be inserted through the collar 364 of delimiter 360, and delimiter 360 may be advanced over the cannula 236 until the delimiter is seated on the housing hub 231, with delimiter arms 361 inserted into apertures (not shown) in housing 210, and with delimiter indicator peg 368 adjacent markings 232 on the housing 210. Then the cannula 236 may be passed over the needle distal end 254 and over the suture holder 240. Force may be applied to the cannula 236, in a proximal direction, such that the cannula proximal end 237 is inserted into the delimiter collar 364.

It will be appreciated by persons skilled in the art that, optionally, assembly of portions of system 200 may be performed in a different order, with the final assembly being identical to that arrived at according to the assembly procedure indicated above. Optionally, assembly of some of the components such as, for example, the locking element, may be omitted, if desired.

### Operation of the System

Operation of system 200 will be described with reference to components of the system discussed hereinabove. It may be noted that the system has three operating positions, namely, retracted, neutral, and advanced, and transition between these operating positions is actuated, in the embodiment shown, by rotation of roller 212, for example, as discussed hereinbelow. In the retracted position, pusher element 230 is retracted to its most proximal position within device 204. In the advanced position, pusher element 230 is advanced to its most distal position within device 204, and may, optionally, extend partly outside the distal end of needle 250 of device 204. In the neutral position, pusher element 230 is disposed halfway between its retracted and advanced positions.

Reference is now made to FIG. 17A-D, which show an exemplary multiple anchor delivery system 200, according to some embodiments of the invention, in an initial operative orientation, e.g., prior to deployment of anchors 270 and 280. As indicated above, the system 200 may include handle 202 having housing 210. Rack 220 may be positioned within housing, with support portions 218a-d (FIGS. 8A-E) supported between flanges 292a-b (FIGS. 7A-E) in housing 210. In the initial operative orientation, in some embodiments, rack 220 may be positioned in a neutral position, e.g., not advanced distally and not retracted, such that projections 229a (FIGS. 8A-D) are seated in middle recess 222a (e.g., FIG. 7A) of housing 210.

As noted above, pusher element 230 may be disposed within needle 250 and pusher element may extend into housing 210 such that pusher element proximal end 326 may be disposed in and retained in bore 227 in the distal end 226 (FIG. 8A) of rack 220. Needle 250 may be disposed within cannula 236, with needle proximal end 252 retained within housing 210 by clamping bars 297 (FIG. 7B) of housing 210, at indentations 257 (FIGS. 13B-C) on needle 250.

Handle 202, pusher element 230, needle 250, and cannula 236 are optionally all arranged along a mutual longitudinal axis 209 (FIG. 17D).

Delimiter 360 may be positioned on the housing distal end 215 such that delimiter hub 362 (FIG. 12A) may be seated on housing hub 231 (FIG. 7A), and cannula 236 may be retained within collar 364 of needle delimiter 360. Delimiter slider 366 may be positioned adjacent markings 232 on housing 210 which may indicate a distance that the needle 250 protrudes out of cannula 236.

Distal movement of slider 366 optionally affects a corresponding distal movement of cannula 236 relative to needle 250, thereby potentially possibly extending cannula 236 further over the needle which can result in less of needle distal end 254 extending out of the cannula distal end 235. Similarly, proximal movement of slider 366 optionally affects a corresponding proximal movement of cannula relative to needle 250, thereby possibly partially withdrawing the cannula 236 from over the needle 250 which can result in more of the needle distal end 254 extending out of the cannula distal end 235. It should be noted that the penetration depth of needle 250 into a tissue 206 of a patient is optionally limited to the extent to which the needle projects distally with respect to the distal end 235 of cannula 236, because the cannula optionally has a blunt distal end and does not enter into the tissue.

While, in the embodiment shown, system 200 includes roller 212c, it will be understood by persons skilled in the art that any of the exemplary rollers 112a-c may be utilized in the device discussed herein. However, for the sake of simplicity, the ensuing description refers to the roller by reference no. 112, to gripping portion as 384, and to operative portion as 380.

Roller 212 may be rotatably mounted on roller mounting peg 299 (FIG. 7B) such that roller 212 resides partially within the housing 210, and extends out of an opening 211 in housing 210. The roller 212 has a gripping portion 384, as noted above, to be contacted by the finger of the user, and an operative portion 380 for interaction with teeth 225 of rack 220.

It is a particular feature of some embodiments of the present invention that, in the initial operative orientation, locking element 342 may be supported against the rack 220, for example, as discussed hereinbelow, which prevents proximal displacement of the rack 220 and, thereby potentially prevents movement of the roller 212 in a counterclockwise direction (toward the needle distal end 254). This may provide a safety feature which may prevent inadvertent or accidental deployment of anchor 270 or anchor 280 from the system 200. In this initial operative orientation, the roller 212 can optionally only be moved in a clockwise direction (away from the needle distal end 254), which may not be intuitive for the user, thereby potentially preventing inadvertent or accidental premature deployment of anchors 270/280.

The rack 220 may be supported by support portions 218a-d between flanges 292a-b of housing inner wall 290 (FIG. 7A-B), such that rack 220 may be slidable along lower flange 292b. Flexible strips 229 extend out of the sides of rack 220, as noted above with regard to FIGS. 8A-D, such that v-shaped projections 229a of the rack may project into any one of recesses 222a-c in each housing portion 210L-R. In the configuration shown in FIG. 17A, rack 220 may be positioned such that projections 229a project into recesses 222b on either side of housing 220.

Teeth 225 of rack 220 may be engaged with corresponding teeth 382 of roller 212 such that rotation of roller 212 in a clockwise direction optionally affects movement of rack 220 in a distal direction. Conversely, rotation of roller 212 in a counterclockwise direction optionally affects movement of rack 220 in a proximal direction. It may be noted that, in the initial operative orientation, roller 212 may be in a neutral position, as indicated by marking 390N being visible on roller 212, in FIG. 17D.

In the initial operative orientation shown, locking bar 346 of locking element 342 has been inserted into recess (FIG. 8D) in rack 220, and body 343 of locking element 342 has been mounted on peg 294 (FIG. 7A) of housing 210 such that locking bar 346 extends vertically upward from body 343 and abuts surface 228b of recess 228 in rack 220. In the initial operative orientation shown, small arm 348 of locking element 342 is positioned below stop 295a (FIG. 7B) of housing 210.

In the embodiment shown, system 200 may be provided with a first solid anchor 270 and a second, hollow anchor 280, the first and second anchors optionally having suture material threaded therethrough, for example, as discussed hereinabove with regard to FIGS. 16A-C. In the initial operative orientation shown, first anchor 270 may be positioned distal to pusher element 230 within needle 250, optionally adjacent the needle distal end 254, and second anchor 280 may be mounted on the pusher element. In the embodiment shown, pusher element 230 extends through second anchor 280. However, other embodiments are conceivable such as, for example, a c-shaped anchor, a solid anchor, or other configurations, for example, as discussed herein and as will be understood by persons skilled in the art. Optionally, the first anchor 270 may abut the distal end 324 of the pusher 230.

According to the embodiment shown, for example, in FIG. 17B, suture portions 288a-b may extend from anchors 270 and 280, out through needle slot 255. It may be noted that the provision of slot 255, which allows the suture portions 288a-b to extend out of the needle, may prevent loops 288a-b from interfering with movement of anchors 270 and 280 through the needle. Suture free end 288c may extend proximally from proximal end 272 of first anchor 270, alongside needle 250, inside optional suture holder 240, and through hub 231 of housing 210. In embodiments where the suture holder 240 is omitted, suture free end 288c may extend proximally alongside needle, inside cannula 236, and through housing hub 231. In the initial operative orientation shown, suture free end 288c had been threaded through bore 306 in bobbin 300 (FIGS. 10A-D) and wound around bobbin spool 302 before bobbin 300 was mounted in housing 210, for example, as discussed herein with regard to FIGS. 7A-B. It will be appreciated by persons skilled in the art that, alternatively, suture free end 288c may be housed within housing 210 without being wound around bobbin 300, may be stored on another component within or outside housing, or may extend at least partly outside of housing.

It may be noted that FIG. 17B shows distal end 242 of suture holder 240 at a location proximal to distal end 235 of cannula. Suture holder 240 may retain a portion of loops 288a-b and suture free end 288c adjacent the needle 250 within the suture holder. Optionally, in some embodiments, suture holder 240 may have a length long enough so that its distal end 242 is closer to the cannula distal end 235 than as configured in FIG. 17B, or approximately flush with cannula distal end 235, thereby potentially retaining a larger portion of loops 288a-b and free end 288c.

Prior to insertion of the needle distal end 254 through tissue 206, a probe is optionally used, for example, of a type known in the art, optionally to measure the thickness of the tissue through which the needle must penetrate. Slider 366 on the delimiter 360 may be advanced or retracted, as desired, until the indicator peg 368 indicates the length of needle distal end 254 which may be insertable through tissue 206. This causes the cannula 236 to be correspondingly advanced or retracted over the needle 250, so that the length of the needle extending out of the cannula is adjusted according to the thickness of the tissue 206 that was measured, as known in the art.

With reference to FIGS. 18A-D, the needle distal end 254 may be inserted through a tissue 206, optionally with the aid of the sharpened tip 253 (FIG. 18C) of needle 250. Due to the presence of cannula 236, only the portion of needle 250 protruding out of cannula distal end 235 may be inserted through tissue 206, the needle being inserted through the tissue 206 at most until cannula 236 abuts the surface of tissue 206.

Optionally, anchor 270 may close off the opening at the needle distal tip 253 and needle slot 255, so that tissue does not collect in the openings. Optionally, first anchor 270 includes an inclined distal end having a configuration that corresponds to that of the needle distal tip 253.

It should be noted that, in this position, although the first anchor 270 may be disposed at least partly through tissue 206, the first anchor has optionally not yet been deployed from the needle 250 and may be still positioned within the needle 250.

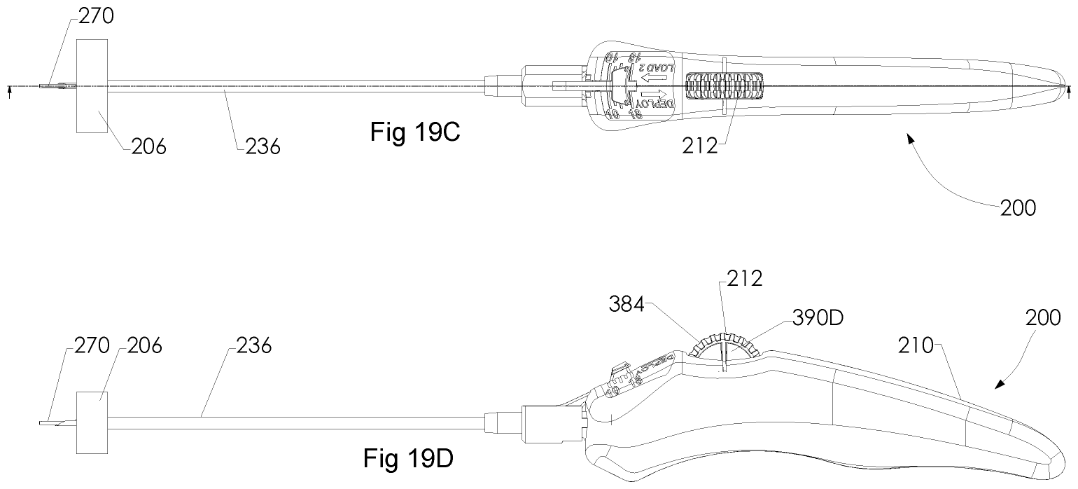

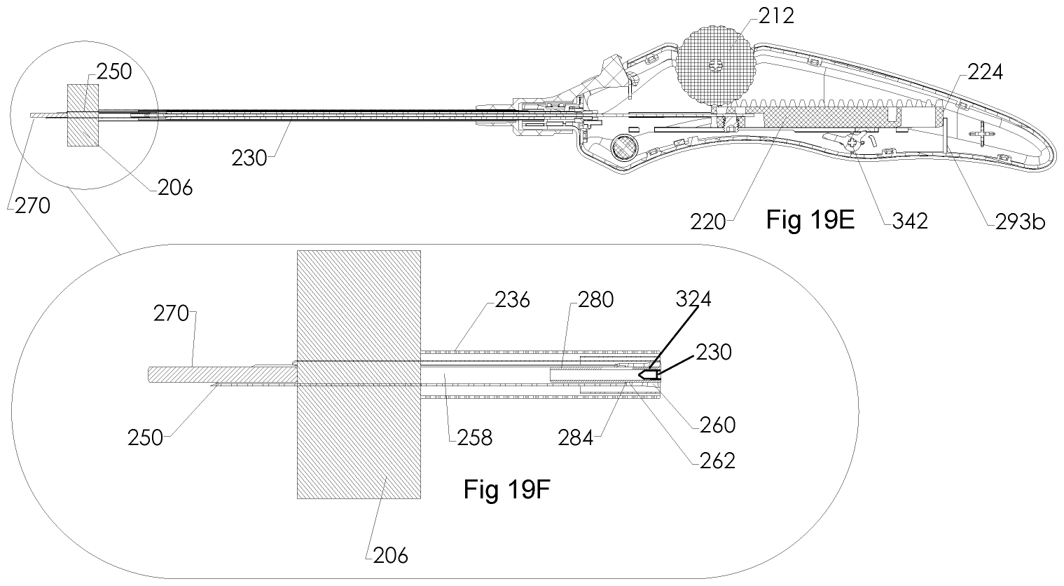

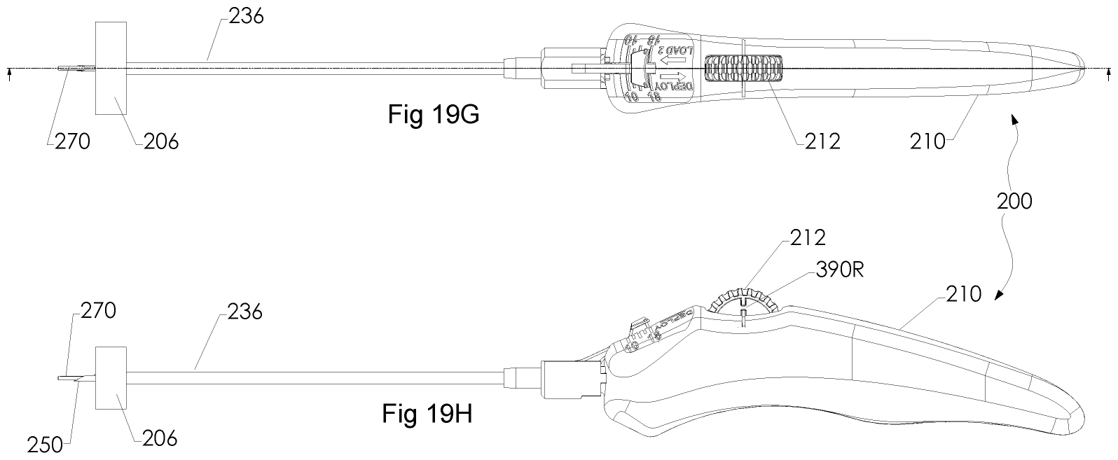

Referring now to FIGS. 19A-D, it should be noted that, in accordance with some embodiments, the components of the system 200 are designed so as to provide the system with the desired aspects discussed herein. For example, in order for the roller to be rotated, a user must displace the gripping portion 384 by a distance sufficient to cause rotational movement of the roller in the desired direction and for the desired degree of rotation, and to move the rack 220 in the desired direction and for the desired distance. This latter movement also requires that the projections 229a (FIGS. 8A-D) on the rack 220 be moved over projections 296a and/or 296b (FIG. 7A) in housing 210.

It may be noted that the amount of force required to rotate the roller 212 in a clockwise direction, from the neutral position (FIG. 17D) is optionally the same as the amount force required to rotate the roller in a counterclockwise direction, from the neutral position. Alternatively, if desired, the amount of force required to rotate the roller 212 in a clockwise direction (to deploy anchor 270) may be more than the amount force required to rotate the roller in a counterclockwise direction (to retract the pusher element 230), or vice versa.

Deployment of first anchor 270 may be actuated by rotation of the roller 212 in a clockwise direction, from a neutral position, in which marking 390N may be visible on roller 212 (FIG. 18D) to a deployed position in which marking 390D may be visible on roller 212, as illustrated in FIG. 19D. This clockwise rotation of the roller 212, optionally affects distal movement of the rack 220, due to the engagement of roller teeth 382 (FIGS. 9A-C) with teeth 225 (FIG. 8A) on rack 220, for example, as discussed herein. This distal movement of rack 220 is accompanied by a corresponding distal movement of the pusher element 230 which is attached to the rack distal end 226. As rack 220 advances distally, the pusher element 230 may be advanced distally to adjacent needle distal end 254, and optionally past the needle distal end, to force the first anchor 270 out of the needle 250 and through tissue 206, as shown in FIGS. 19A-D.

It may be noted that, during deployment of the first anchor 270, the proximal end 272 of the first anchor is at a location distal to the second anchor 280.

As noted above, clockwise rotation of roller 212 can optionally affect a corresponding movement of the rack 220 in a distal direction. As the rack 220 moves distally, from the position shown in FIG. 18A to the position shown in FIG. 19A, support portions 218a-d (FIGS. 8A-E) slide distally along housing flanges 292a-b (FIGS. 7A-B). The distal movement of rack 220 is sufficient to move the v-shaped projections 229a on rack 220 from recesses 222b, over projections 296a, and into recesses 222a on inner walls 290 of housing portions 210L-R. Further, movement of the projections 229a over the projections 296a optionally may cause an audible indication such as, for example, a clicking sound, and/or a tactile indication, which informs a user that the first anchor has been deployed. Optionally and/or alternatively, an electronic circuit may be provided to indicate to the user that the first anchor 270 has been deployed.

It may be noted that, while in the embodiment shown in FIGS. 19A-22C first and second anchors 270/280 are illustrated as straight elements, this is for illustrative purposes only. It will be appreciated by person skilled in the art that, optionally, once deployed, each of anchors 270/280 may have a different configuration such as, for example, a curve, depending on rigidity of the anchor and the amount of force exerted on it by the suture portions 288a-c extending through tissue 206.

As noted above, as the roller 212 is rotated in a clockwise direction, the rack 220 begins to move distally, e.g., from the position shown in FIG. 18A to the position shown in FIG. 19A. At the same time, the locking element 342 begins to rotate in a counterclockwise direction due to the locking bar 346 being pushed by surface 228b (FIG. 8D) of the rack 220, as the rack moves distally. At the same time, the small arm 348 of locking element 342 begins to slide along stop 295a (FIG. 7B) in housing 210. As the rack 220 continues to move distally, the locking element 342 continues to rotate as the locking bar 364 moves down the surface 228a of the rack 220, toward the opening of the recess 228, and the small arm 348 continues to slide along stop 295a. Once rack 220 has moved distally enough so that the locking bar 364 is adjacent the opening of the recess 228, the small arm 348 is adjacent knob 295b at the end of stop 295a. Further movement of the rack 220 pushes the locking bar 364 further until it has exited the recess 228. Optionally, at the same time, small arm 348 is pushed over the knob 295b, optionally producing a slight clicking noise. This slight clicking noise should not be confused with the clicking noise created by movement of projections 229a on rack over the projections 296a in housing 210, noted above. The small arm 348 is prevented from moving further, as it is now positioned between knob 295b and stop 295c. At this point the locking element is disengaged from the rack 220. From this point on, the locking element 342 will remain in this position, regardless of the position of the rack 220.

It may be noted that, once the roller 212 has been rotated clockwise, to the position shown in FIG. 19D, further clockwise rotation of roller 212 may be prevented due to the distal end 226 of rack 220 abutting a stop 293a provided on housing 210. As rack 220 cannot move further distally, this prevents further rotation of roller 212 in a clockwise direction, due to the engagement of teeth 225 of the rack 220 with teeth 382 of the roller 212.

With particular reference to FIG. 19B, an anchor stopper 260 may be located within the interior volume 258 of needle 250, optionally at the proximal portion of the needle 250. The anchor stopper 260 defines a distally facing surface 262. Anchor stopper 260 defines a barrier in the needle 250 whereat the passageway 264 is narrowed, thereby potentially obstructing possible proximal movement of the second anchor 280 within the passageway. The proximal end 284 of the second anchor 280 is optionally disposed in the vicinity or abuts the distally facing surface 262 of the anchor stopper 260. Alternatively, the proximal end 284 of the second anchor 280 may be slightly distally spaced apart from distally facing surface 262 of the anchor stopper 260.

After deployment of the first anchor 270, the proximal end 284 of the second anchor 280 is optionally more distally spaced apart from distally facing surface 262 of the anchor stopper 260 than in the initial operative orientation (FIG. 17B). This may be due to its having been displaced distally by distal movement of the pusher element as the first anchor is deployed.

Reference is now made to FIGS. 19E-H, wherein the system 200 is shown in a retraction operative orientation. Roller 212 has been rotated in a counterclockwise direction, from the position shown in FIG. 19D to the position shown in FIG. 19H, in which marking 390R may be visible on roller 212. This counterclockwise rotation of roller 212 optionally affected proximal movement of rack 220, due to the engagement of roller teeth 382 with teeth 225 on rack, for example, as discussed herein with reference to FIGS. 9A-C. This proximal movement of rack 220 was optionally accompanied by a corresponding proximal movement of the pusher element 230 which is attached to the rack distal end 226. As rack 220 moves proximally, the pusher element 230 may be retracted within needle 250.

It should be noted that, even in the most retracted position of the pusher element 230, the pusher element proximal end 326 remains within the housing 210. Optionally and alternatively, pusher element 230 may extend proximally out of the housing, for example, up to 5 cm or more than 5 cm.

As noted above, counterclockwise rotation of roller 212 optionally affects a corresponding movement of the rack 220 in a proximal direction. As the rack 220 moves proximally, from the position shown in FIG. 19A to the position shown in FIG. 19E, support portions 218a-d (FIGS. 8A-E) slide proximally along housing flanges 292a-b (FIG. 7A-B). The proximal movement of rack 220 may be sufficient to move the v-shaped projections 229a on rack 220 from recesses 222a, over projections 296a and 296b, and into recesses 222c on inner walls 290 of housing portions 210L-R. Further, movement of the projections 229a over the projections 296a and 296b optionally may cause an audible indication such as, for example, a single or a double clicking sound, and/or a tactile indication, which informs a user that the pusher element 230 has been retracted and that the second anchor 280 has been loaded and may be ready for deployment. Optionally and/or alternatively, an electronic circuit may be provided to indicate to the user that the pusher element 230 has been retracted.

It is a further particular feature of some embodiments of the present invention that in this retraction operative orientation, the locking element 342 remains disengaged from the rack 220, and does not prevent further advancement or retraction of the pusher element 230. However, roller 212 may be prevented from rotating further in a counterclockwise direction due to the proximal end 224 of rack 220 abutting a stop 293b provided on housing 210. As rack 220 cannot move further proximally, this optionally prevents further rotation of roller 212 in a counterclockwise direction, due to the engagement of teeth 225 of the rack 220 with teeth 382 of the roller 212.

It is seen in FIG. 19F that in this retraction operative orientation, the first anchor 270 remains deployed outside of the needle 250.

It is a particular feature of some embodiments of the present invention that, during proximal retraction of the pusher 230, from the position in FIGS. 19A-B to the position in FIGS. 20A-B, the second anchor 280 may be released from the pusher 230 into the interior volume 258 of the needle 250, optionally within suture holder 240, and may not be mounted over the pusher 230 anymore. Rather the proximal end 284 of the second anchor 280 optionally abuts and/or optionally is supported against distally facing surface 262 of the anchor stopper 260 in this retraction operative orientation, thus preventing proximal displacement of the second anchor 280 past the anchor stopper 260.

It is seen in FIG. 19F that the distal end 324 of the pusher 230 may now be proximally spaced from the proximal end 284 of the second anchor 280.

With reference to FIGS. 20A-D, system 200 is shown still in the retracted orientation, after needle 250 has been withdrawn from tissue 206. Roller 212 is still in the position shown in FIG. 20E, in which marking 390R may be visible on roller 212.

It is seen in FIGS. 20A-B that, as the first anchor 270 has been deployed outside of the needle 250 and second anchor 280 remains inside the needle, suture portions 288a-c extend from anchor 270, on one side of tissue 206, through the tissue, to anchor 280 which is still inside needle 250 (FIG. 20B).

It should be noted that, for example, as described above with regard to FIGS. 19E-20D, after first anchor 270 has been deployed, retraction of pusher element 230 was followed by withdrawal of the needle from tissue 206. Alternatively, the order of these operations may be reversed, so that first needle 250 may be withdrawn from tissue 206 and then pusher element 230 may be retracted.

Optionally, it may be desired to implant second anchor 280 at the same location on tissue 206, using an anchor configuration known in the art as a “vertical mattress.” In this case, needle 250 may be reinserted into tissue 206 at the same location as that at which it was inserted for deployment of first anchor 270. Alternatively, it may be desired to implant second anchor 280 at a different location from that at which first anchor 270 was deployed, using an anchor configuration known in the art as “horizontal mattress.” In this case, needle 250 may be inserted into tissue 206 at a different location from that at which it was inserted for deployment of first anchor 270. According to the embodiment described below with reference to FIGS. 20E to 22C, first and second anchors 270 and 280 are deployed in the horizontal mattress configuration.

With reference to FIGS. 20E-G, when it is desired to deploy second anchor 280 through tissue 206, needle 250 is again inserted through tissue 206. System 200 is shown still in the retracted orientation. It may be noted that roller marking 390R (FIG. 20D) may still be shown on roller 212 to indicate that the pusher element 230 has been retracted.

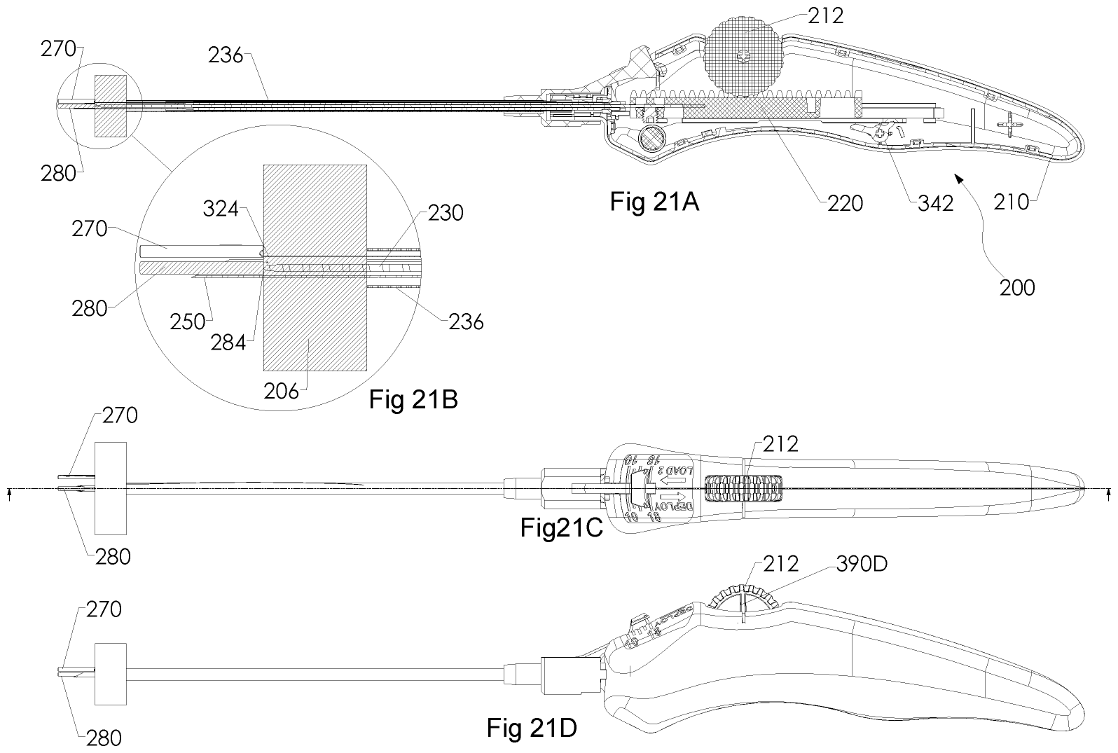

Reference is now made to FIG. 21A-D, which illustrates a second anchor deployment operative orientation, in accordance with some embodiments. In this configuration, deployment of second anchor 280 has been actuated by rotation of the roller 212 in a clockwise direction, from the retracted position, in which marking 390R may be visible on roller 212 (FIG. 20D) to a deployed position in which marking 390D may be visible on roller 212, as illustrated in FIG. 21D.

Clockwise rotation of the roller 212 optionally affected distal movement of the rack 220, due to the engagement of roller teeth 382 with teeth 225 on rack 220, for example, as discussed herein with regard to FIGS. 9A-C. This distal movement of rack 220 was accompanied by a corresponding distal movement of the pusher element 230 which is attached to the rack distal end 226. As rack 220 advances distally, the pusher element 230 may be advanced distally to adjacent the needle distal end 254, to force the second anchor 280 out of the needle 250. The second anchor 280 deployed through tissue 206 may be seen, for example, in FIG. 21A-D.

As noted above, with regard to deployment of first anchor 270, movement of components within device 204 may optionally cause an audible indication such as, for example, a double clicking sound, and/or a tactile indication, which informs a user that the second anchor 280 has been deployed. Optionally and/or alternatively, an electronic circuit may be provided to indicate to the user that the second anchor 280 has been deployed. It is a further particular feature of some embodiments of the present invention that in this second anchor deployment operative orientation, the locking element 342 remains disengaged from the rack 220. However, from the position of roller shown in FIG. 21E, further clockwise rotation of roller 212 may be prevented, for example, as described herein in detail with regard to FIG. 19A-D.

It is seen in FIG. 21D that in this second anchor deployment operative orientation, the second anchor 280 has been optionally pushed distally due to distal displacement of the pusher 230 and engagement of distal end 324 of pusher 230 with the proximal end 284 of the second anchor 280. The second anchor 280 may now be disposed distally with respect to the distal end 254 of the needle 250 and may now be deployed out of the interior volume 258 of the needle 250 and through tissue 206 of the patient.

With regard to FIGS. 18A-19H and 21A-D, it should be noted that, optionally, needle distal end 254 may be inserted through tissue 206 so that it exits the tissue by a distance at least large enough so that the entire length of anchors 270 and 280 is deployed through the tissue. Optionally and alternatively, if distal tip 253 only of needle 250 were to penetrate tissue 206, with a portion of needle distal end 254 still within the tissue, anchors 270/280 may not entirely exit the tissue.

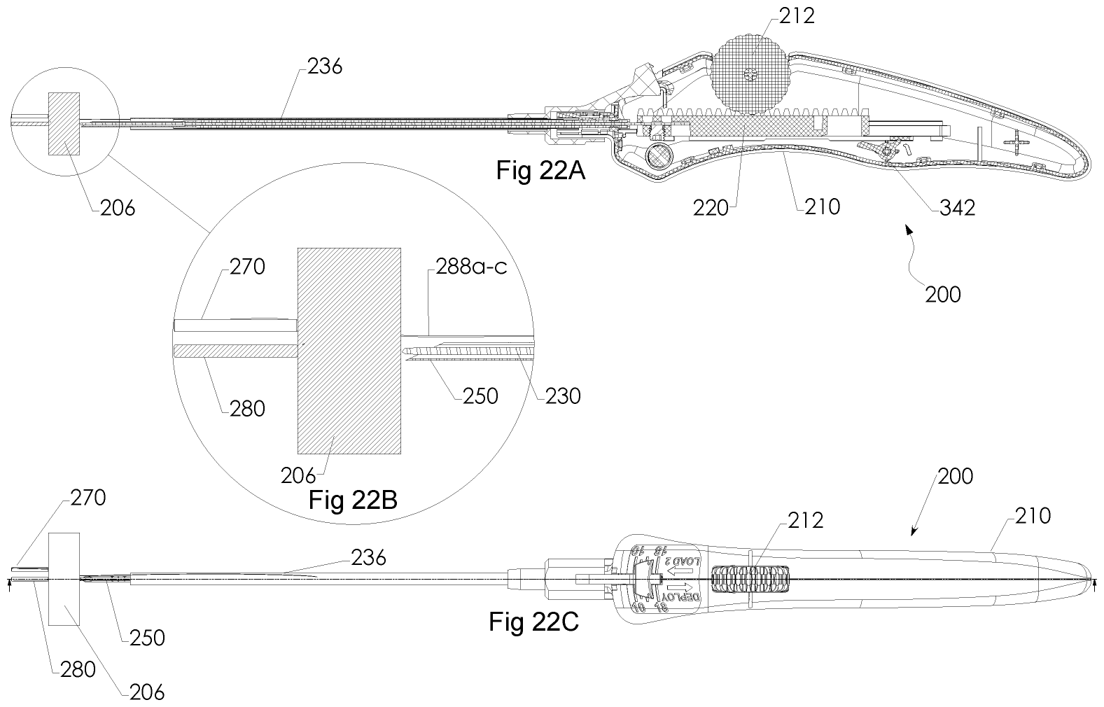

With reference to FIG. 22A-C there is shown the system 200, in accordance with some embodiments of the invention, after second anchor 280 has been deployed (FIGS. 21A-D), and after the device 204 has been moved away from tissue 206, whereby the needle 250 has been withdrawn from the tissue, as seen most clearly in FIG. 22B.

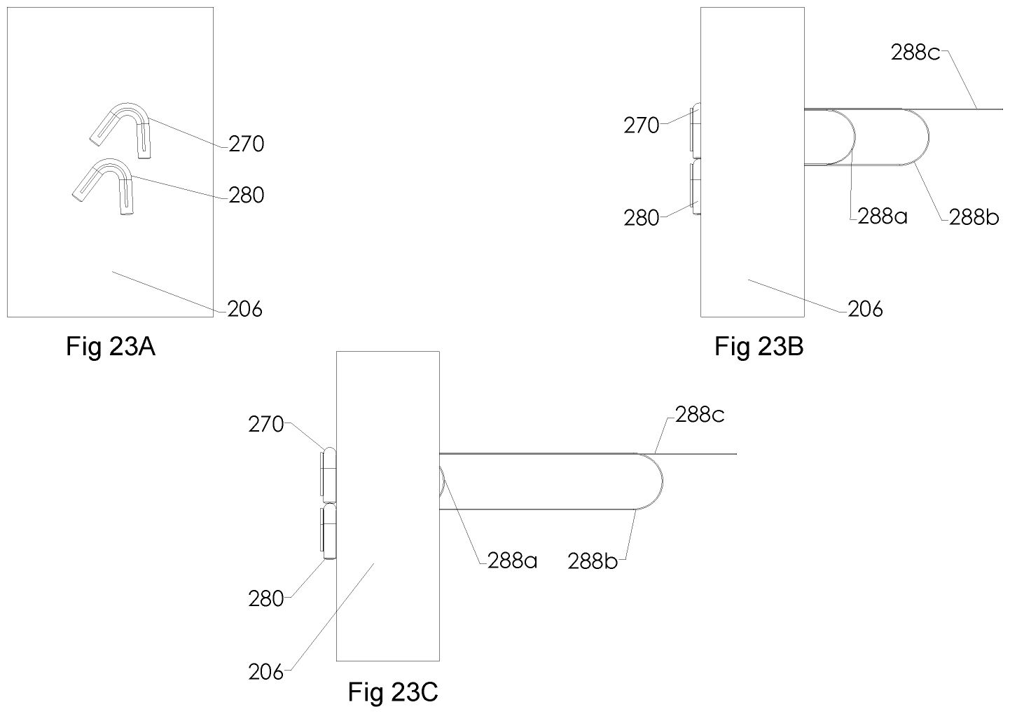

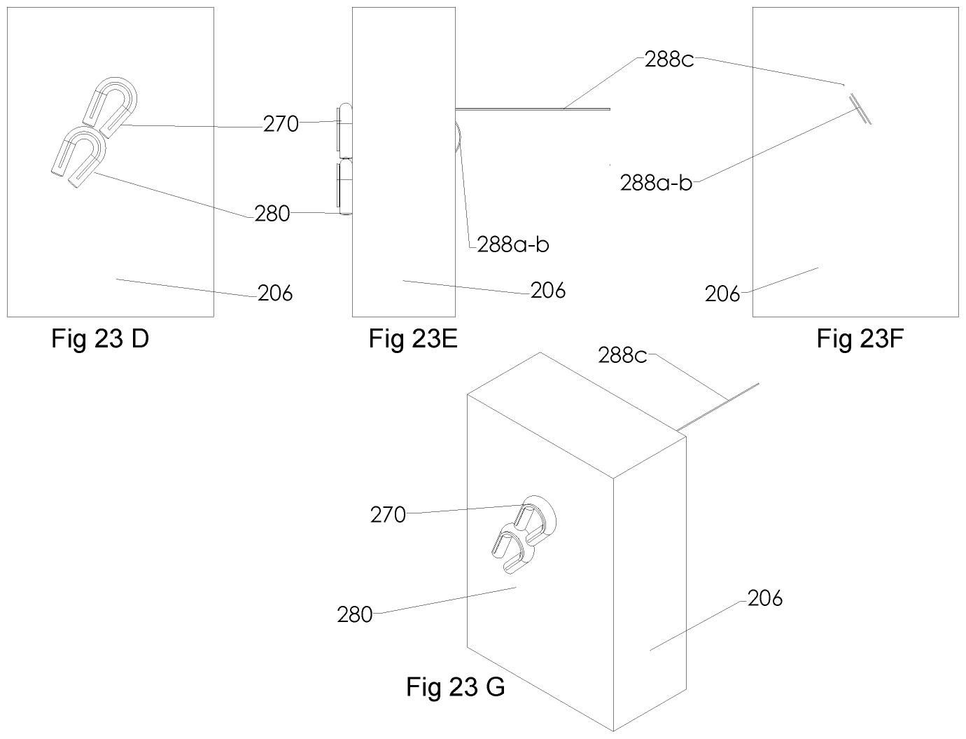

It should be noted that, in this configuration, the suture portions 288a-c which are attached to the first and second anchors, have been released from the device and may extend through tissue 206 to its proximal side. Although suture portions 288a-c appear in FIG. 22B as a single line, it will be understood by persons skilled in the art that ref nos. 288a-c in this drawing represents small loop 288a, large loop 288b, and suture free end 288c. The suture material may optionally be tightened to secure the first and second anchors 270/280 to the tissue and thereby potentially anchor portions of the tissue together, for example, as described in detail below with reference to FIGS. 23A-G.

With reference to FIGS. 23A-G there is shown tightening of first anchor 270 and second anchor 280 against tissue 206, after deployment of the anchors from device 204. Anchors 207 and 280 are shown in FIGS. 23A-B, where they are loosely held in position adjacent one side of the tissue 206 with the suture material including small and large loops 288a-b and suture free end 288c on the opposite side of the tissue.

While in the embodiment shown, anchors 270 and 280 optionally appear as being generally u-shaped, alternatively, the anchors may each appear as straight or slightly curved into a c-shape, depending, for example, on the rigidity of the anchors and how tightly the anchors are pulled by the suture material.

In order to tightly fasten the anchors 270 and 280 against tissue 206, the long suture portion 288b may be pulled. This shortens the small loop 288a, which causes each of the anchors to assume a narrow u-shape and to be tightened against tissue 206, as shown in FIGS. 23C-D. Then the suture free end 288c may be pulled by the user. This shortens the large loop 288b, as shown in FIGS. 23E-G. As the suture free end 288c was previously twisted around the large loop 288b, this pulling of the free end creates a knot in the suture material as the large loop 288b gets smaller. The excess suture length may then be cut off, optionally using a cutting device such as, for example, described in U.S. Pat. No. 6,866,673.

In accordance with an alternative embodiment, both a first anchor and a second anchor may be loaded together in parallel into a sheath, and each of the anchors may be optionally covered by a resilient cover. Both anchors may be loaded together into a single needle and a driving assembly may be provided, having a first rack coupled to a first pusher that deploys the first anchor 270 and a second rack coupled to a second pusher that deploys the second anchor 280. Optionally, when the first rack is advanced distally to deploy the first anchor, the second rack is in a lag mode. Optionally, when the first rack is retracted, the second rack is advanced distally to deploy the second anchor.

Yet further, in accordance with another alternative embodiment, the system may include more than two anchors. For example, there may be provided a single solid anchor, deployable, for example, as described above with regard to FIGS. 19A-D, and two or more tubular anchors mounted on a pusher element and deployable by the pusher element. Optionally, depending on how far back the pusher element is retracted, more than one tubular anchor may be deployed with the same distal displacement of the pusher element.

Optionally, the anchors in accordance with any of the described embodiments may or may not be attached by suture material. It is noted that the system 200 may be configured to be disposable.

### Methods of the Invention

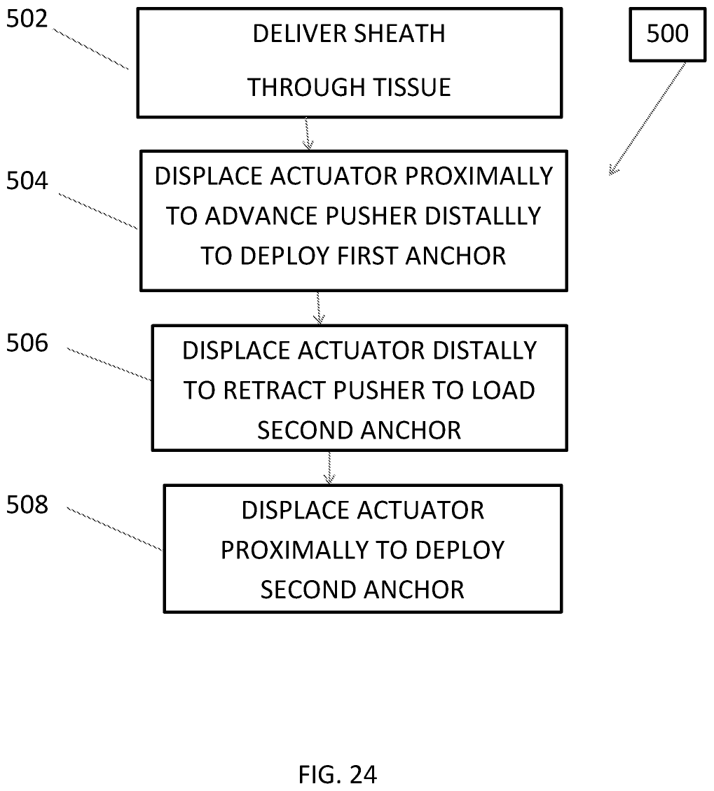

With reference to FIG. 24, there is shown a method 500 of delivering multiple anchors into the tissue of a patient, in accordance with a particular aspect of some embodiments of the present invention. At 502, a sheath may be delivered through a tissue, for example, as discussed herein, sheath may be, for example, a hollow needle, optionally having a sharpened tip. At 504, an actuator may be displaced in a proximal direction to deploy a first anchor. For example, as discussed herein, displacement of the actuator may be, for example, linear displacement of a trigger or rotational movement of a roller. Thereafter, at 506, the actuator may be displaced in a distal direction to load a second anchor; and thereafter, at 508, another displacement of the roller in the proximal direction to deploy the second anchor 280.

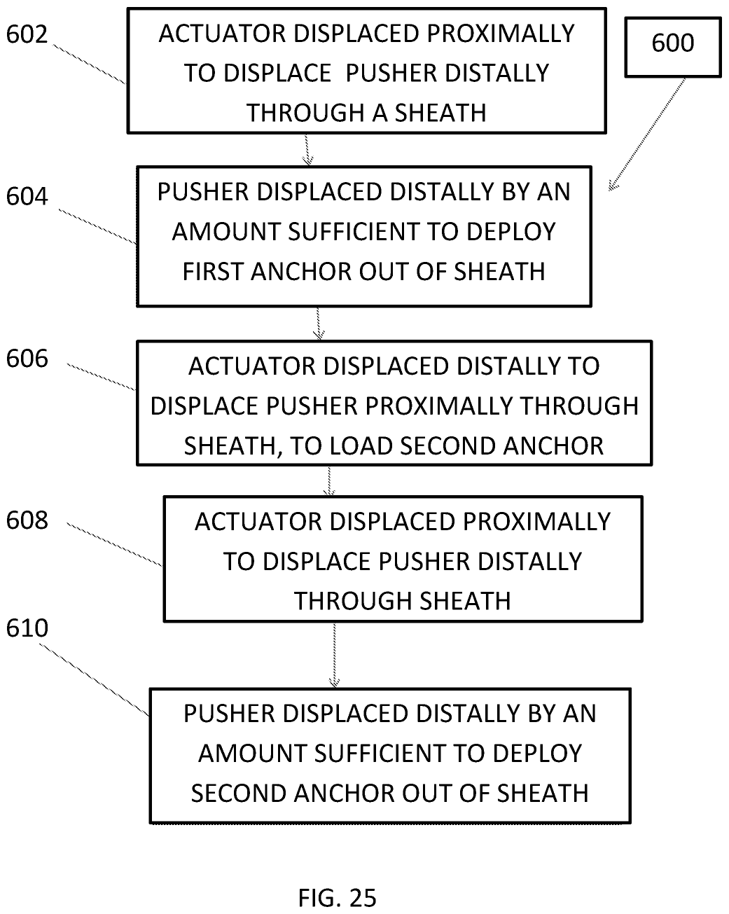

With reference to FIG. 25, it is a particular feature of some embodiments of the present invention that the method 600 of operation of a device for delivering multiple anchors into the tissue of a patient optionally includes, at 602, proximal displacement of an actuator in order to displace a pusher distally through a sheath. For example, as discussed herein, displacement of the actuator may be, for example, linear displacement of a trigger or rotational movement of a roller. Simultaneously, at 604, the pusher is displaced distally by an amount sufficient to deploy the first anchor out of the sheath. Thereafter, at 606, distal displacement of the actuator causes the pusher to be moved proximally, which results in loading of a second anchor. Thereafter, at 608, displacement of the roller in the proximal direction causes the pusher to be moved distally through the sheath. Simultaneously, at 610, the pusher is displaced distally by an amount sufficient to deploy the second anchor 280 out of the sheath.

It should be noted that the device may be operated outside the body, for example, in accordance with the actions indicated above, and/or not during a medical procedure, such as, for example, during testing of the device.

It is expected that during the life of a patent maturing from this application many relevant anchoring systems will be developed and the scope of the term anchor is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A multiple anchor delivery system, comprising:
a sheath having a proximal end and a distal end and having a passageway extending therethrough;
a pusher element having at least a distal end positioned within said passageway, said pusher element distal end sized and shaped to be displaced through said passageway;
a first anchor disposed within said sheath, said first anchor having a proximally-facing end surface, said first anchor proximally-facing end surface positioned distally with respect to said pusher element; and
a second anchor disposed within said sheath, proximally with respect to said first anchor, said second anchor disposed within said passageway and, said second anchor sized and shaped to be displaced along said passageway;
said pusher element sized and shaped to be displaced in a distal direction, wherein said pusher element is configured to abut said first anchor proximally-facing end surface, and wherein said distal displacement of said pusher element has a first displacement length sufficient to deploy said first anchor;
said pusher element sized and shaped to be displaced in a proximal direction to a position proximal to at least a distal portion of said second anchor; and
said displacement of said pusher element having a second displacement length at least as long as the distance between said proximal end of said second anchor and said sheath distal end, said second displacement length sufficient to deploy said second anchor.

2-4. (canceled)

5. The system according to claim 1, wherein said second anchor is tubular, said system further including a barrier that narrows said passageway, said barrier obstructing a proximal displacement of said second anchor.

6-9. (canceled)

10. The system according to claim 1, wherein said second anchor is compressible in a lateral direction, said pusher element displaceable in a proximal direction to a position proximal to said second anchor.

11. The system according to claim 1, wherein said first anchor includes a blind bore at its proximal end, said pusher element distal end sized and shaped to fit into said blind bore.

12. (canceled)

13. The system according to claim 1, wherein said pusher element distal end includes a retaining portion for temporarily retaining said second anchor thereon, wherein said retaining portion is a recess for retaining a second anchor.

14. The system according to claim 1, wherein said pusher element is provided with a compressible distal portion.

15-17. (canceled)

18. The system according to claim 1, wherein said sheath has a lateral opening at the distal end thereof, said opening large enough to for at least two suture elements to pass therethrough.

19. The system according to claim 1, wherein said sheath has a lateral opening at the distal end thereof, said second anchor positioned proximal to said lateral opening prior to deployment of said second anchor.

20-23. (canceled)

24. The system of claim 1, further including:
an actuator mechanically coupled to said pusher element, movement of said actuator affecting linear displacement of said pusher element along said passageway; and
a driving unit for displacing said pusher element through said passageway, wherein said actuator is mechanically coupled with said driving unit;
wherein displacement of said actuator in a distal direction affects via said driving unit said displacement of said pusher element in a proximal direction.

25. (canceled)

26. The system according to claim 1, further including a housing for housing said pusher element and at least a proximal portion of said sheath, said housing having a semicircular portion at a distal end thereof and a suture holder having a semicircular arm at a proximal end thereof, said arm and said housing portion together forming a passageway sized for passage therethrough of a suture.

27. The system according to claim 1, further comprising a locking element for preventing said proximal displacement of said pusher element.

28. The system according to claim 27, further comprising:
a driving unit for displacing said pusher element through said passageway;
wherein said locking element obstructs a path of movement of said driving unit.

29. (canceled)

30. A multiple anchor delivery system, comprising:
a sheath having a proximal end and a distal end and having a passageway extending therethrough;
first and second pusher elements disposed inside said sheath, each pusher element having at least a distal end positioned within said passageway, said distal end of each said pusher element sized and shaped to be displaced through said passageway;
a first anchor disposed within said sheath, said first anchor having a proximally-facing end surface, at least said proximally-facing end surface of said first anchor positioned distally with respect to said first pusher element;
a second anchor disposed within said sheath, proximally with respect to said first anchor;
said first pusher element displaceable in a distal direction, into contact with said first anchor proximally-facing end surface, said displacement having a displacement length sufficient to deploy said first anchor;
said second pusher element displaceable in a distal direction, into contact with said second anchor, said displacement having a displacement length sufficient to deploy said second anchor.

31. A multiple anchor delivery system, comprising:
a sheath having a proximal end and a distal end and having a passageway extending therethrough;
a pusher element having at least a distal end positioned within said passageway, said pusher element distal end sized and shaped to be displaced through said passageway;
a first anchor disposed within said sheath, said first anchor having a proximally-facing end surface, said proximally-facing end surface of said first anchor positioned distally with respect to said pusher element, wherein said pusher element is configured to abut said first anchor proximally-facing end surface; and
a second anchor disposed within said sheath, proximally with respect to said first anchor, said second anchor mounted on said pusher element.

32-34. (canceled)

35. A method of delivering multiple anchors into a tissue of a patient, comprising:
delivering a sheath through a tissue such that a sheath distal end penetrates the tissue;
displacing an actuator in a proximal direction to apply a force to a proximally-facing end surface of a first anchor to deploy the first anchor out of the sheath and through the tissue;
displacing the actuator in a distal direction to position a proximal end of a pusher element at least partly proximally to said second anchor; and
displacing the actuator in said proximal direction to engage the second anchor and to deploy the second anchor out of the sheath.

36. The method of claim 35, wherein said first anchor is provided with a suture portion, said suture portion deployed out of said sheath and through the tissue with deployment of said first anchor.

37. A method of operation of a device for deployment of multiple anchors, comprising:
first displacement of a pusher element through a sheath in a distal direction;
engagement of the pusher element with a proximally-facing end surface of a first anchor located in the sheath, said first displacement having a first displacement length sufficient to deploy the first anchor out of the sheath;
displacement of the pusher element through the sheath in a proximal direction, to a position proximal to a second anchor;
second displacement of the pusher element through the sheath in the distal direction; and
engagement of the pusher element with the second anchor during said second displacement, said second displacement having a second displacement length sufficient to deploy said second anchor out of the sheath.

38. The system according to claim 1, wherein said proximally-facing end surface of said first anchor is preformed.

39. The system according to claim 1, wherein said first anchor is a solid anchor.

40. A multiple anchor delivery system, comprising:
a sheath having a proximal end and a distal end and having a passageway extending therethrough, said sheath including a recess adjacent said passageway;
a pusher element having at least a distal end positioned within said passageway, said pusher element distal end sized and shaped to be displaced through said passageway;
a first anchor disposed within said sheath, at least a portion of said first anchor positioned distally with respect to said pusher element; and
a second anchor disposed within said recess of said sheath, said pusher element interfering with positioning of said second anchor in said passageway said second anchor having a proximal end, said second anchor sized and shaped to be displaced along said passageway;
said pusher element sized and shaped to be displaced in a proximal direction to a position proximal to said second anchor, thereby no longer interfering with positioning of said second anchor in said passageway; and
said pusher element sized and shaped to be displaced in a distal direction, said displacement of said pusher element in said distal direction, from said position proximal to said second anchor, bringing said pusher element into contact with said proximal end of said second anchor, said displacement of said pusher element in the distal direction having a displacement length at least as long as the distance between said proximal end of said second anchor and said sheath distal end, said displacement length sufficient to deploy said second anchor.
